(19) United States
(12) Patent Application Publication
(10) Pub. No.: US 2005/0169927 A1
(43) Pub. Date: Aug. 4, 2005

(54) Human tissue factor antibodies

(75) Inventors: Per-Ola Freskgaard, Vellinge (SE); Jes Clausen, Hong (DK); Brit Binow Sorensen, Birkerod (DK); Marianne Kjalke, Kobenhavn (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/095,423

(22) Filed: Mar. 31, 2005

Related U.S. Application Data
(63) Continuation of application No. 10/261,706, filed on Sep. 30, 2002, now abandoned.

(30) Foreign Application Priority Data
Oct. 2, 2001 (DK) PA 2001 01437

(51) Int. Cl. C12P 21/04; A61K 39/395; C07K 16/40

(52) U.S. Cl. 424/146.1; 435/70.21; 530/388.26

(57) ABSTRACT

The present invention relates to isolated fully human antibodies that immunoreacts with human tissue factor (TF) to inhibit the binding of coagulation factor VIIa (FVIIa).

### CROSS-REFERENCE TO RELATED APPLICATIONS

This application is a continuation of U.S. patent application Ser. No. 10/261,706, filed Sep. 30, 2002, and claims priority under 35 U.S.C. § 119 of Danish Patent Application No. PA 2001 01437, filed Oct. 2, 2001, and U.S. Provisional Patent Application No. 60/329,775, filed Oct. 16, 2001, the contents of all of which are hereby fully incorporated herein by reference.

### FIELD OF THE INVENTION

The present invention relates to isolated antibodies that immunoreacts with tissue factor (TF) to inhibit the binding of coagulation factor VIIa (FVIIa) and thus an immunotherapeutic method using human antibodies against TF to inhibit thrombus formation associated with surgery, microsurgery, angioplasty or trauma or to inhibit thrombus formation and other functions of TF in abnormal haemostatic conditions associated with diseases like deep vein thrombosis, disseminated intravascular coagulation (DIC), coronary artery disease, sepsis, inflammation, atherosclerosis, or cancer. Also disclosed are a method for preparation of antibodies as well as cell lines for preparation of the human monoclonal antibodies (Mabs).

### BACKGROUND OF THE INVENTION

Blood coagulation is a process consisting of a complex interaction of various blood components, or factors, which eventually gives rise to a fibrin clot. Generally, the blood components which participate in what has been referred to as the coagulation “cascade” are proenzymes or zymogens, enzymatically inactive proteins which are converted to proteolytic enzymes by the action of an activator, itself an activated clotting factor. Coagulation factors that have undergone such a conversion and generally referred to as “active factors,” and are designated by the addition of a lower case “a” suffix (e.g., Factor VIIa).

Activated Factor X (“Xa”) is required to convert prothrombin to thrombin, which then converts fibrinogen to fibrin as a final stage in forming a fibrin clot. There are two systems, or pathways, that promote the activation of Factor X. The “intrinsic pathway” refers to those reactions that lead to thrombin formation through utilization of factors present only in plasma. A series of protease-mediated activations ultimately generates Factor IXa which, in conjunction with Factor VIIIa, cleaves Factor X into Xa. An identical proteolysis is effected by FVIIa and its co-factor, TF, in the “extrinsic pathway” of blood coagulation. TF is a membrane bound protein and does not normally circulate in an active form in plasma. Upon vessel disruption, however, TF can complex with FVIIa to catalyze Factor X activation or Factor IX activation in the presence of Ca²⁺ and phospholipid. While the relative importance of the two coagulation pathways in hemostasis is unclear, Factor VII and TF have been found to play a pivotal role in the initiation of blood coagulation.

It is often necessary to selectively block the coagulation cascade in a patient. Anticoagulants such as heparin, coumarin, derivatives of coumarin, indandione derivatives, or other agents may be used, for example, during kidney dialysis, or to treat deep vein thrombosis, disseminated intravascular coagulation (DIC), and a host of other medical disorders. For example, heparin treatment or extracorporeal treatment with citrate ion may be used in dialysis to prevent coagulation during the course of treatment. Heparin is also used in preventing deep vein thrombosis in patients undergoing surgery.

Treatment with heparin and other anticoagulants may, however, have undesirable side effects. Available anticoagulants generally act throughout the body, rather than acting specifically at site of injury. Heparin, for example, may cause heavy bleeding. Furthermore, with a half-life of approximately 80 minutes, heparin is rapidly cleared from the blood, necessitating frequent administration. Because heparin acts as a cofactor for antithrombin III (ATIII), and ATIII is rapidly depleted in DIC treatment, it is often difficult to maintain the proper heparin dosage, necessitating continuous monitoring of ATIII and heparin levels. Heparin is also ineffective if ATIII depletion is extreme. Further, prolonged use of heparin may also increase platelet aggregation and reduce platelet count, and has been implicated in the development of heparin-induced thrombocytopenia. Indandione derivatives may also have toxic side effects. In addition to the anticoagulants briefly described above, several naturally occurring proteins have been found to have anticoagulant activity. Also, ATIII has been proposed as a therapeutic anticoagulant.

International Application No. WO 92/15686 relates to inactivated Factor VIIa for inhibiting blood coagulation.

Antibodies are specific immunoglobulin (Ig) polypeptides produced by the vertebrate immune system in response to challenges by foreign proteins, glycoproteins, cells, or other antigenic foreign substances. The sequence of events which permits the organism to overcome invasion by foreign cells or to rid the system of foreign substances is at least partially understood. An important part of this process is the manufacture of antibodies which bind specifically to a particular foreign substance. The binding specificity of such polypeptides to a particular antigen is highly refined, and the multitude of specificities capable of being generated by the individual vertebrate is remarkable in its complexity and variability. Millions of antigens are capable of eliciting antibody responses, each antibody almost exclusively directed to the particular antigen which elicited it.

Two major sources of vertebrate antibodies are presently utilized, generation in situ by the mammalian B lymphocytes, and generation in cell culture by B-cell hybrids. Antibodies are generated in situ as a result of the differentiation of immature B lymphocytes into plasma cells, which occurs in response to stimulation by specific antigens. In the undifferentiated B cells, the portions of DNA coding for the various regions on the immunoglobulin chains are separated in the genomic DNA. The sequences are assembled sequentially prior to expression. The resulting rearranged gene is capable of expression in the mature B lymphocyte to produce the desired antibody. However, even when a particular mammal is exposed to only a single antigen a uniform population of antibodies does not result. The in situ immune response to any particular antigen is defined by the mosaic of responses to the various determinants which are present on the antigen. Each subset of homologous antibodies is contributed by a single population of B cells, hence in situ generation of antibodies is “polyclonal”.

This limited but inherent heterogeneity has been overcome in numerous particular cases by use of hybridoma technology to create “monoclonal” antibodies in cell cultures by B cell hybridomas.

In this process, the relatively short-lived, or mortal, splenocytes or lymphocytes from a mammal which has been injected with antigen are fused with an immortal tumor cell line, thus producing hybrid cells or “hybridomas” which are both immortal and capable of producing the genetically coded antibody of the B cell. The hybrids thus formed are segregated into single genetic strains by selection, dilution, and regrowth, and each strain thus represents a single genetic line. They therefore, produce antibodies which are assured to be homogeneous against a desired antigen. These antibodies, referencing their pure genetic parentage, are called “monoclonal”.

Monoclonal antibodies with mono-specificity have greatly influenced immunology, and their usefulness has already been demonstrated in such sciences as biology, pharmacology, biochemistry and others. Such monoclonal antibodies have found widespread use not only as diagnostics reagents, but also therapeutically (see, for example, Ritz and Schlossman, Blood, 59:1-11, (1982)).

Monoclonal antibodies produced by hybridomas, while theoretically effective as discussed above and clearly preferable to polyclonal antibodies because of their specificity, suffer from an important disadvantage. In many applications, the use of monoclonal antibodies produced in non-human animals is severely restricted where the monoclonal antibodies are to be used in humans. Repeated injections of a “foreign” antibody in humans, such as a mouse antibody, may lead to harmful hypersensitivity reactions. Such a non-human derived monoclonal antibody, when injected into humans, causes an anti-nonhuman antibody response.

Therapeutic use of mouse Mabs against TF is known from U.S. Pat. Nos. 6,001,978 and 5,223,427.

International Application No. WO 99/51743 relates to human/mouse chimera monoclonal antibodies directed against human TF.

European patent application No. 833911 relates to CDR-grafted antibodies against human TF.

Presta L. et al., Thrombosis and Haemostasis, Vol. 85 (3) pp. 379-389 (2001) relates to humanized antibody against TF.

There is still a need in the art for improved compositions having anticoagulant activity which can be administered at relatively low doses and do not produce the undesirable side effects associated with traditional anticoagulant compositions. The present invention fulfils this need by providing anticoagulants that do not have the side effects associated with the traditional antibodies with non-human sequences, they act specifically at sites of injury, and further provides other related advantages. Furthermore the present invention provides compounds, which acts to inhibit the cellular functions of TF, which is implicated in conditions like sepsis, inflammation, atherosclerosis, restenosis, or cancer.

### DESCRIPTION OF THE INVENTION

The present invention relates to non-immunogenic high affinity human antibodies against human TF, which inhibits the binding of coagulation factor VII/VIIa and methods for selection of therapeutically effective human antibodies against human TF.

In a first aspect, the present invention relates to an isolated human antibody, which immunoreacts with an epitope present on human TF.

The terms “human tissue factor” or “human TF” as used herein, refers to the full length polypeptide receptor comprising the amino acid sequence 1-263 of native human tissue factor.

The term “antibody”, as used herein, is intended to refer to immunoglobulin molecules and fragments thereof, that have the ability to specifically bind to an antigen (e.g., human TF). Full-length antibodies comprises four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Thus, within the definition of an antibody is also one or more fragments of an antibody that retain the ability to specifically bind to an antigen (e.g., human TF). It has been shown that the antigen-binding function of an antibody can be performed by fragments of a full-length antibody. Examples of binding fragments encompassed within the term “antibody” include (i) a Fab fragment, a monovalent fragment consisting of the VL, VH, CL and CH I domains; (ii) F(ab)₂ and F(ab′)₂ fragments, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426: and Huston et al. (1988) Proc. Natl. Acad. Sci. USA 85:5879-5883). Such single chain antibodies are also intended to be encompassed within the term “antibody”. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poijak, R. J., et al. (1994) Structure 2:1121-1123). It is understood that human TF may have one or more antigenic determinants comprising (1) peptide antigenic determinants which consist of single peptide chains within human TF, (2) conformational antigenic determinants which consist of more than one spatially contiguous peptide chains whose respective amino acid sequences are located disjointedly along the human TF polypeptide sequence; and (3) post-translational antigenic determinants which consist, either in whole or part, of molecular structures covalently attached to human TF after translation, such as carbohydrate groups, or the like.

The terms “human antibody”, “human antibodies”, “human TF antibody”, and “human TF antibodies”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences, e.g. the so-called humanized antibodies or human/mouse chimera antibodies.

An “isolated human antibody”, as used herein, is intended to refer to a human antibody that is substantially free of other antibodies having different antigenic specificities (e.g., an isolated antibody that specifically binds human TF is substantially free of antibodies that specifically bind antigens other than human TF). An isolated antibody that specifically binds human TF may, however, have cross-reactivity to other antigens, such as TF molecules from other species (discussed in further detail below). Moreover, an isolated antibody may be substantially free of other cellular material and/or chemicals.

The term “epitope” as used herein means any antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

The terms “immunoreacts” or “immunoreacting”, as used herein, means any binding of an antibody to its epitope with a dissociation constant K_{d} lower than 10⁻⁴ M. The terms “immunoreacts” or “immunoreacting” are used where appropriate interchangeably with the term “specifically bind”.

The term “inhibits”, as used herein, means any reduction compared to a reference. As an example, an antibody, which inhibits the binding of human coagulation factor VIIa to human TF means any antibody, which reduces the ability of human coagulation factor VIIa to bind human TF compared to the ability of human coagulation factor VIIa to bind human TF in the absense of the antibody.

The term “affinity”, as used herein, means the strength of the binding of an antibody to an epitope. The affinity of an antibody is measured by the dissociation constant K_{d}, defined as [Ab]×[Ag]/[Ab−Ag] where [Ab−Ag] is the molar concentration of the antibody-antigen complex, [Ab] is the molar concentration of the unbound antibody and [Ag] is the molar concentration of the unbound antigen. The affinity constant Kₐ is defined by 1/K_{d}. Preferred methods for determining Mabs specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference.

In a second aspect, the invention relates to a pharmaceutical composition comprising a therapeutically effective amount of a human antibody, which immunoreacts with an epitope present on human TF.

The term “a therapheutically effective amount” is the effective dose to be determined by a qualified practitioner, who may titrate dosages to achieve the desired response. Factors for consideration of dose will include potency, bioavailability, desired pharmacokinetic/pharmacodynamic profiles, condition of treatment (e.g. trauma, inflammation, septic chock), patient-related factors (e.g. weight, health, age, etc.), presence of co-administered medications, time of administration, or other factors known to a medical practitioner. The dosage of a human antibody against TF administered to a patient will vary with the type and severity of the condition to be treated, but is generally in the range of 0.1-5.0 mg/kg body weight.

The term “subject” as used herein is intended to mean any animal, in particular mammals, such as humans, and may, where appropriate, be used interchangeably with the term “patient”.

In a third aspect, the invention relates to a composition comprising a human antibody, which immunoreacts with an epitope present on human TF.

In a further aspect, the invention relates to a method for treatment of a FVIIa/TF related disorder in a human, which method comprises administering to the human a therapeutically effective amount of a human antibody, which immunoreacts with an epitope present on human TF.

“Treatment” means the administration of an effective amount of a therapeutically active compound of the invention with the purpose of preventing any symptoms or disease state to develop or with the purpose of curing or easing such symptoms or disease states already developed. The term “treatment” is thus meant to include prophylactic treatment.

The terms “FVIIa/TF related disorder” as used herein means a disease or disorder, where TF and FVIIa are involved. Included are thrombotic or coagulopathic related diseases or disorders including inflammatory response and chronic thromboembolic diseases or disorders associated with fibrin formation including vascular disorders such as deep venous thrombosis, arterial thrombosis, post surgical thrombosis, coronary artery bypass graft (CABG), percutaneous transdermal coronary angioplastry (PTCA), stroke, tumor growth, tumor metastasis, angiogenesis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases or disorders. The FVIIa/TF related disorder is not limited to in vivo coagulopatic disorders such as those named above but includes ex vivo FVIIa/TF related processes such as coagulation that may result from the extracorporeal circulation of blood, including blood removed in-line from a patient in such processes as dialysis procedures, blood filtration, or blood bypass during surgery.

The term “Factor VIIa”, or “FVIIa” means “two chain” activated coagulation factor VII cleaved by specific cleavage at the Arg152-Ile153 peptide bond. FVIIa, may be purified from blood or produced by recombinant means. It is evident that the practice of the methods described herein is independent of how the purified factor VIIa is derived and, therefore, the present invention is contemplated to cover use of any factor VIIa preparation suitable for use herein. Preferred are human FVIIa.

The term “FVII” means “single chain” coagulation factor VII.

In a further aspect, the invention relates to a method for preparation of a human antibody, which method comprises
a) Preparation of human antibodies against human TF,
b) testing antibodies in a TF-induced clot assay and selecting human antibody which inhibit clot formation in this assay with an IC₅₀ value lower than 1 nM, such as lower than 500 pM, preferably lower than 200 pM, preferably lower than 100 pM, preferably lower than 50 pM, preferably lower than 10 pM, more preferably lower than 5 pM, or
   testing antibodies in a FXa generation assay and selecting human antibody which inhibit FXa generation with an IC₅₀ value lower than 100 nM (in an assay with a FVIIa concentration of 0.1 nM), such as lower than 10 nM, preferably lower than 5 nM, preferably lower than 1 nM, more preferably lower than 0.1 nM, or
   testing antibodies in a FVIIa/TF amidolytic assay and selecting human antibody which inhibit TF-induced FVIIa amidolytic activity, with an IC₅₀ value lower than 100 nM (in an assay with a FVIIa concentration of 10 nM), such as lower than 40 nM, preferable lower than 20 nM, more preferably lower than 10 nM, or
   testing antibodies in a FVIIa competition assay and selecting human antibody which compete with FVIIa binding, or
   testing antibodies in a TF ELISA assay comprising TF and selecting human antibody which bind human TF.

It is to be understood, that the specific IC₅₀ values referred to in the TF-induced clot assay is when using normal human plasma.

In a further aspect, the invention relates to a method for preparation of a human antibody, which method comprises
a) Preparation of human antibodies against human TF,
b) testing antibodies in a TF-induced clot assay and selecting human antibody which inhibit clot formation in this assay with an IC₅₀ value lower than the IC₅₀ value of FFR-rFVIIa+1 nM, such as lower than the IC₅₀ value of FFR-rFVIIa+500 pM, preferably lower than the IC₅₀ value of FFR-rFVIIa+200 pM, preferably lower than the IC₅₀ value of FFR-rFVIIa+100 pM, preferably lower than the IC₅₀ value of FFR-rFVIIa+50 pM, preferably lower than the IC₅₀ value of FFR-rFVIIa+10 pM, more preferably lower than the IC₅₀ value of FFR-rFVIIa+5 pM, more preferably lower than the IC₅₀ value of FFR-rFVIIa, or
   testing antibodies in a FXa generation assay and selecting human antibody which inhibit FXa generation with an IC₅₀ value lower than the IC₅₀ value of FFR-rFVIIa+100 nM (using 0.1 nM FVIIa in the assay), such as lower than the IC₅₀ value of FFR-rFVIIa+10 nM, preferably lower than the IC₅₀ value of FFR-rFVIIa+5 nM, preferably lower than the IC₅₀ value of FFR-rFVIIa+1 nM, more preferably lower than the IC₅₀ value of FFR-rFVIIa+0.1 nM, more preferably lower than the IC₅₀ value of FFR-rFVIIa, or
   testing antibodies in a FVIIa/TF amidolytic assay and selecting human antibody which inhibit TF-induced FVIIa amidolytic activity, with an IC₅₀ value lower than the IC₅₀ value of FFR-rFVIIa+100 nM (using 10 nM FVIIa in the assay), such as lower than the IC₅₀ value of FFR-rFVIIa+40 nM, preferably lower than the IC₅₀ value of FFR-rFVIIa+20 nM, preferably lower than the IC₅₀ value of FFR-rFVIIa+10 nM, more preferably lower than the IC₅₀ value of FFR-rFVIIa, or
   testing antibodies in a FVIIa competition assay and selecting human antibody which compete with FVIIa binding, or
   testing antibodies in a TF ELISA assay comprising TF and selecting human antibody which immunoreacts with human TF.

It is to be understood, that the specific IC₅₀ values referred to in the TF-induced clot assay is when using normal human plasma.

The term “TF-induced clot assay” as used herein is intended to mean any assay where clotting time is measured in sample comprising blood plasma and TF. An example of a TF-induced clot assay is described in example 1, assay 7.

The term “FXa generation assay” as used herein is intended to mean any assay where activation of FX is measured in a sample comprising TF, FVIIa, FX, calcium and phospholipids. An example of a FXa generation assay is described in example 1, assay 5.

The term “FVIIa/TF amidolytic assay” as used herein is intended to mean any assay where the amidolytic activity, i.e. cleavage of a small peptide substrate, of FVIIa is measured in the presence of TF. An example of a FVIIa/TF amidolytic assay is described in example 1, assay 4.

The term “TF ELISA assay” as used herein is intended to mean any ELISA assay comprising TF and antibodies against TF. Examples of TF ELISA assays are the direct and indirect TF ELISA assays described in example 1, assay 1 and 2.

The term “direct TF ELISA assay” as used herein is intended to mean any TF ELISA assay comprising immobilized TF. Example of direct TF ELISA assays is described in example 1, assay 1.

The term “indirect TF ELISA assay” as used herein is intended to mean any TF ELISA assay, where TF is in solution. Example of direct TF ELISA assays is described in example 1, assay 2.

In a further aspect, the invention relates to a human antibody which immunoreacts with an epitope present on human TF and inhibits the binding of human coagulation factor VIIa to human TF obtainable by a method comprising:
a) Preparation of human antibodies against human TF,
b) testing antibodies in a TF-induced clot assay and selecting human antibody which inhibit clot formation in this assay with an IC₅₀ value lower than 1 nM, such as lower than 500 pM, preferably lower than 200 pM, preferably lower than 100 pM, preferably lower than 50 pM, preferably lower than 10 pM, more preferably lower than 5 pM, or
   testing antibodies in a FXa generation assay and selecting human antibody which inhibit FXa generation with an IC₅₀ value lower than 100 nM (in an assay with a FVIIa concentration of 0.1 nM), such as lower than 10 nM, preferably lower than 5 nM, preferably lower than 1 nM, more preferably lower than 0.1 nM, or
   testing antibodies in a FVIIa/TF amidolytic assay and selecting human antibody which inhibit TF-induced FVIIa amidolytic activity, with an IC₅₀ value lower than 100 nM (in an assay with a FVIIa concentration of 10 nM), such as lower than 40 nM, preferable lower than 20 nM, preferably lower than 10 nM, or
   testing antibodies in a FVIIa competition assay and selecting human antibody which compete with FVIIa binding, or
   testing antibodies in a TF ELISA assay comprising TF and selecting human antibody which bind human TF.

It is to be understood, that the specific IC₅₀ values referred to in the TF-induced clot assay is when using normal human plasma.

In a further aspect, the invention relates to a human antibody which immunoreacts with an epitope present on human TF and inhibits the binding of human coagulation factor VIIa to human TF obtainable by a method comprising:
a) Preparation of human antibodies against human TF,
b) testing antibodies in a TF-induced clot assay and selecting human antibody which inhibit clot formation in this assay with an IC₅₀ value lower than the IC₅₀ value of FFR-rFVIIa+1 nM, such as lower than the IC₅₀ value of FFR-rFVIIa+500 pM, preferably lower than the IC₅₀ value of FFR-rFVIIa+200 pM, preferably lower than the IC₅₀ value of FFR-rFVIIa+100 pM, such as lower than the IC₅₀ value of FFR-rFVIIa+50 pM, preferably lower than the IC₅₀ value of FFR-rFVIIa+10 pM, more preferably lower than the IC₅₀ value of FFR-rFVIIa+5 pM, more preferably lower than the IC₅₀ value of FFR-rFVIIa, or
   testing antibodies in a FXa generation assay and selecting human antibody which inhibit FXa generation with an IC₅₀ value lower than the IC₅₀ value of FFR-rFVIIa+100 nM (using 0.1 nM FVIIa in the assay), such as lower than the IC₅₀ value of FFR-rFVIIa+10 nM, preferably lower than the IC₅₀ value of FFR-rFVIIa+5 nM, preferably lower than the IC₅₀ value of FFR-rFVIIa+1 nM, more preferably lower than the IC₅₀ value of FFR-rFVIIa+0.1 nM, more preferably lower than the IC₅₀ value of FFR-rFVIIa, or
   testing antibodies in a FVIIa/TF amidolytic assay and selecting human antibody which inhibit TF-induced FVIIa amidolytic activity, with an IC₅₀ value lower than the IC₅₀ value of FFR-rFVIIa+100 nM (using 10 nM FVIIa in the assay), such as lower than the IC₅₀ value of FFR-rFVIIa+40 nM, preferably lower than the IC₅₀ value of FFR-rFVIIa+20 nM, preferably lower than the IC₅₀ value of FFR-rFVIIa+10 nM, more preferably lower than the IC₅₀ value of FFR-rFVIIa, or
   testing antibodies in a FVIIa competition assay and selecting human antibody which compete with FVIIa binding, or
   testing antibodies in a TF ELISA assay comprising TF and selecting human antibody which immunoreacts with human TF.

It is to be understood, that the specific IC₅₀ values referred to in the TF-induced clot assay is when using normal human plasma.

In one embodiment of the invention the method, wherein the human antibodies against human TF are produced comprises immunization of a mammal with human TF, and isolation of antibodies produced by the immunized mammal. In a prefered embodiment, the mammal is a mouse. It is to be understod, that the immunized mammal or mouse is capable of producing human antibodies.

In a further aspect, the invention relates to a method for preparation of a human antibody, which method comprises
a) immunization of mouse with human TF,
b) isolation of antibody-producing cell from immunized mouse and preparation of immortal cells to secrete human antibodies,
c) isolation of culture medium from immortal cells comprising produced antibodies,
d) testing antibodies in an indirect TF ELISA assay comprising TF in solution and selecting human antibodies which binds human TF in solution,
e) testing antibodies in a FVIIa competition assay and selecting human antibodies which competes with FVIIa binding,
f) testing antibodies in a FVIIa/TF amidolytic assay and selecting human antibodies which inhibits TF-induced FVIIa amidolytic activity, with an IC₅₀ value lower than 100 nM (in an assay with a FVIIa concentration of 10 nM), such as lower than 40 nM, preferable lower than 20 nM, more preferably lower than 10 nM,
g) testing antibodies in a FXa generation assay and selecting human antibodies which inhibits FXa generation with an IC₅₀ value lower than 100 nM (in an assay with a FVIIa concentration of 0.1 nM), such as lower than 10 nM, preferably lower than 5 nM, preferably lower than 1 nM, more preferably lower than 0.1 nM,
h) testing antibodies in a TF-induced clot assay and selecting human antibody which inhibit clot formation in this assay with an IC₅₀ value lower than 1 nM, such as lower than 500 pM, preferably lower than 200 pM, preferably lower than 100 pM, preferably lower than 50 pM, preferably lower than 10 pM, more preferably lower than 5 pM,
i) selection and cultivation in a suitable culture medium of the immortal cell producing the selected antibody following step d-h,
j) isolation of selected antibody from culture medium of selected immortal cell.

It is to be understood, that the specific IC₅₀ values referred to in the TF-induced clot assay is when using normal human plasma.

In a further aspect, the invention relates to a method for preparation of a human antibody, which method comprises
a) immunization of mouse with human TF,
b) isolation of antibody-producing cell from immunized mouse and preparation of immortal cells to secrete human antibodies,
c) isolation of culture medium from immortal cells comprising produced antibodies,
d) testing antibodies in an indirect TF ELISA assay comprising TF in solution and selecting human antibodies which immunoreacts with human TF in solution,
e) testing antibodies in a FVIIa competition assay and selecting human antibodies which competes with FVIIa binding,
f) testing antibodies in a FVIIa/TF amidolytic assay and selecting human antibody which inhibit TF-induced FVIIa amidolytic activity, with an IC₅₀ value lower than the IC₅₀ value of FFR-rFVIIa+100 nM (using 10 nM FVIIa in the assay), such as lower than the IC₅₀ value of FFR-rFVIIa+40 nM, preferably lower than the IC₅₀ value of FFR-rFVIIa+20 nM, preferably lower than the IC₅₀ value of FFR-rFVIIa+10 nM, more preferably lower than the IC₅₀ value of FFR-rFVIIa,
g) testing antibodies in a FXa generation assay and selecting human antibody which inhibit FXa generation with an IC₅₀ value lower than the IC₅₀ value of FFR-rFVIIa+100 nM (using 0.1 nM FVIIa in the assay), such as lower than the IC₅₀ value of FFR-rFVIIa+10 nM, preferably lower than the IC₅₀ value of FFR-rFVIIa+5 nM, preferably lower than the IC₅₀ value of FFR-rFVIIa+1 nM, more preferably lower than the IC₅₀ value of FFR-rFVIIa+0.1 nM, more preferably lower than the IC₅₀ value of FFR-rFVIIa,
h) testing antibodies in a TF-induced clot assay and selecting human antibodies which inhibits clot formation in this assay with an IC₅₀ value lower than the IC₅₀ value of FFR-rFVIIa+1 nM, such as lower than the IC₅₀ value of FFR-rFVIIa+500 pM, preferably lower than the IC₅₀ value of FFR-rFVIIa+200 pM, preferably lower than the IC₅₀ value of FFR-rFVIIa+100 pM, such as lower than the IC₅₀ value of FFR-rFVIIa+50 pM, preferably lower than the IC₅₀ value of FFR-rFVIIa+10 pM, more preferably lower than the IC₅₀ value of FFR-rFVIIa+5 pM, more preferably lower than the IC₅₀ value of FFR-rFVIIa,
i) selection and cultivation in a suitable culture medium of the immortal cell producing the selected antibody following step d-h,
j) isolation of selected antibody from culture medium of selected immortal cell.

It is to be understood, that the specific IC₅₀ values referred to in the TF-induced clot assay is when using normal human plasma.

The term “antibody-producing cell” as used herein means any cell capable of producing an antibody. Included are hybridomas, transfected cell lines and the relatively shortlived, or mortal, splenocytes or lymphocytes from a mammal which has been injected with an antigen.

In a further aspect, the invention relates to a human antibody which immunoreacts with an epitope present on human TF and inhibits the binding of human coagulation factor VIIa to human TF obtainable by a method comprising:
a) immunization of mouse with human TF,
b) isolation of antibody-producing cells from immunized mouse and preparation of immortal cells to secrete human antibodies,
c) isolation of culture medium from immortal cells comprising produced antibodies,
d) testing antibodies in an indirect TF ELISA assay comprising TF in solution and selecting human antibodies which binds human TF in solution,
e) testing antibodies in a FVIIa competition assay and selecting human antibodies which competes with FVIIa binding,
f) testing antibodies in a FVIIa/TF amidolytic assay and selecting human antibodies which inhibits TF-induced FVIIa amidolytic activity, with an IC₅₀ value lower than 100 nM (in an assay with a FVIIa concentration of 10 nM), such as lower than 40 nM, preferable lower than 20 nM, more preferably lower than 10 nM,
g) testing antibodies in a FXa generation assay and selecting human antibodies which inhibits FXa generation with an IC₅₀ value lower than 100 nM (in an assay with a FVIIa concentration of 0.1 nM), such as lower than 10 nM, preferably lower than 5 nM, preferably lower than 1 nM, more preferably lower than 0.1 nM,
h) testing antibodies in a TF-induced clot assay and selecting human antibody which inhibit clot formation in this assay with an IC₅₀ value lower than 1 nM, such as lower than 500 pM, preferably lower than 200 pM, preferably lower than 100 pM, preferably lower than 50 pM, preferably lower than 10 pM, more preferably lower than 5 pM,
i) selection and cultivation in a suitable culture medium of the immortal cell producing the selected antibody following step d-h,
j) isolation of selected antibody from culture medium of selected immortal cell.

It is to be understood, that the specific IC₅₀ values referred to in the TF-induced clot assay is when using normal human plasma.

In a further aspect, the invention relates to a human antibody which immunoreacts with an epitope present on human TF and inhibits the binding of human coagulation factor VIIa to human TF obtainable by a method comprising:
a) immunization of mouse with human TF,
b) isolation of antibody-producing cell from immunized mouse and preparation of immortal cells to secrete human antibodies,
c) isolation of culture medium from immortal cells comprising produced antibodies,
d) testing antibodies in an indirect TF ELISA assay comprising TF in solution and selecting human antibodies which immunoreacts with human TF in solution,
e) testing antibodies in a FVIIa competition assay and selecting human antibodies which competes with FVIIa binding,
f) testing antibodies in a FVIIa/TF amidolytic assay and selecting human antibody which inhibit TF-induced FVIIa amidolytic activity, with an IC₅₀ value lower than the IC₅₀ value of FFR-rFVIIa+100 nM (using 10 nM FVIIa in the assay), such as lower than the IC₅₀ value of FFR-rFVIIa+40 nM, preferably lower than the IC₅₀ value of FFR-rFVIIa+20 nM, preferably lower than the IC₅₀ value of FFR-rFVIIa+10 nM, more preferably lower than the IC₅₀ value of FFR-rFVIIa,
g) testing antibodies in a FXa generation assay and selecting human antibody which inhibit FXa generation with an IC₅₀ value lower than the IC₅₀ value of FFR-rFVIIa+100 nM (using 0.1 nM FVIIa in the assay), such as lower than the IC₅₀ value of FFR-rFVIIa+10 nM, preferably lower than the IC₅₀ value of FFR-rFVIIa+5 nM, preferably lower than the IC₅₀ value of FFR-rFVIIa+1 nM, more preferably lower than the IC₅₀ value of FFR-rFVIIa+0.1 nM, more preferably lower than the IC₅₀ value of FFR-rFVIIa,
h) testing antibodies in a TF-induced clot assay and selecting human antibodies which inhibits clot formation in this assay with an IC₅₀ value lower than the IC₅₀ value of FFR-rFVIIa+1 nM, such as lower than the IC₅₀ value of FFR-rFVIIa+500 pM, preferably lower than the IC₅₀ value of FFR-rFVIIa+200 pM, preferably lower than the IC₅₀ value of FFR-rFVIIa+100 pM, such as lower than the IC₅₀ value of FFR-rFVIIa+50 pM, preferably lower than the IC₅₀ value of FFR-rFVIIa+10 pM, more preferably lower than the IC₅₀ value of FFR-rFVIIa+5 pM, more preferably lower than the IC₅₀ value of FFR-rFVIIa,
i) selection and cultivation in a suitable culture medium of the immortal cell producing the selected antibody following step d-h,
j) isolation of selected antibody from culture medium of selected immortal cell.

It is to be understood, that the specific IC₅₀ values referred to in the TF-induced clot assay is when using normal human plasma.

In one embodiment of the invention the immortal cell is a hybridoma cell.

In a further aspect, the invention relates to a cell producing human antibodies which immunoreacts with an epitope present on human TF and inhibits the binding of human coagulation factor VIIa to human TF.

In one embodiment the cell is an isolated lymphoid cell.

In a further embodiment the cell is isolated from a mouse.

In a further embodiment the cell is a hybridoma cell. In one embodiment the the hybridoma cell is obtained by fusion of an antibody-producing lymphoid cell with an immortal cell to provide an antibody-producing hybridoma cell.

In a further embodiment of the invention the isolated human antibody inhibits the binding of human coagulation factor VIIa to human TF.

In a further embodiment of the invention the isolated human antibody is a monoclonal antibody.

The term “monoclonal antibody” as used herein, refers to a homogeneous population of immunoglobulins, i.e. the individual molecules of the antibody population are identical except for naturally occurring mutations. Antibodies are normally synthesized by lymphoid cells derived from B lymphocytes of bone marrow. Lymphocytes derived from the same clone produce immunoglobulin of a single amino acid sequence. Lymphocytes can not be directly cultured over long periods of time to produce substantial amounts of their specific antibody. However, Kohler et al., 1975, Nature, 256:495, demonstrated that a process of somatic cell fusion, specifically between a lymphocyte and a myeloma cell, could yield hybridoma cells which grow in culture and produce a specific antibody called a “monoclonal antibody”. Myeloma cells are lymphocyte tumor cells which, depending upon the cell strain, frequently produce an antibody themselves, although “non-producing” strains are known.

In a further embodiment of the invention the isolated human antibody is a recombinant antibody.

The term “recombinant antibody”, as used herein, is intended to include all human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In a further embodiment of the invention the isolated human antibody is a Fab fragment.

In a further embodiment of the invention the isolated human antibody is a F(ab)₂ fragment.

In a further embodiment of the invention the isolated human antibody is a F(ab′)₂ fragment.

In a further embodiment of the invention the isolated human antibody is a single chain Fv fragment.

In a further embodiment of the invention the isolated human antibody has a K_{d} for binding to human TF within the range of 10⁻¹⁵-10⁻⁸ M. It is to be understood, that the K_{d} for human antibody binding to human TF referred to is as determined in an assay, wherein the human antibody is immobilized (see assay 6).

In a further embodiment of the invention the isolated human antibody has a K_{d} for binding to human TF within the range of 10⁻¹⁵-10⁻¹⁰ M.

In a further embodiment of the invention the isolated human antibody has a K_{d} for binding to human TF lower than 10⁻⁸ M. In a further embodiment of the invention the isolated human antibody has a K_{d} for binding to human TF lower than 10⁻⁹ M In a further embodiment of the invention the isolated human antibody has a K_{d} for binding to human TF lower than 10⁻¹⁰ M. In a further embodiment of the invention the isolated human antibody has a K_{d} for binding to human TF lower than 10⁻¹¹ M. In a further embodiment of the invention the isolated human antibody has a K_{d} for binding to human TF lower than 10⁻¹² M. In a further embodiment of the invention the isolated human antibody has a K_{d} for binding to human TF lower than 10⁻¹³ M. In a further embodiment of the invention the isolated human antibody has a K_{d} for binding to human TF lower than 10⁻¹⁴ M. In a further embodiment of the invention the isolated human antibody has a K_{d} for binding to human TF lower than 10⁻¹⁵ M.

In a further embodiment of the invention the method for preparation of a human antibody comprises testing antibodies in a TF-induced clot assay and selecting human antibody which inhibit clot formation in this assay with an IC₅₀ value lower than 1 nM. In a further embodiment the IC₅₀ value is lower than 500 pM. In a further embodiment the IC₅₀ value is lower than 200 pM. In a further embodiment the IC₅₀ value is lower than 100 pM. In a further embodiment the IC₅₀ value is lower than 50 pM. In a further embodiment the IC₅₀ value is lower than 10 pM. In a further embodiment the IC₅₀ value is lower than 5 pM.

In a further embodiment of the invention the method for preparation of a human antibody comprises testing antibodies in a TF-induced clot assay and selecting human antibody which inhibit clot formation in this assay with an IC₅₀ value lower than the IC₅₀ value of FFR-rFVIIa+1 nM, such as lower than the IC₅₀ value of FFR-rFVIIa+500 pM, preferably lower than the IC₅₀ value of FFR-rFVIIa+200 pM, preferably lower than the IC₅₀ value of FFR-rFVIIa+100 pM, such as lower than the IC₅₀ value of FFR-rFVIIa+50 pM, preferably lower than the IC₅₀ value of FFR-rFVIIa+10 pM, more preferably lower than the IC₅₀ value of FFR-rFVIIa+5 pM, more preferably lower than the IC₅₀ value of FFR-rFVIIa.

It is to be understood, that the specific IC₅₀ values referred to in the TF-induced clot assay is when using normal human plasma.

In a further embodiment of the invention the method for preparation of a human antibody comprises testing antibodies in a FXa generation assay and selecting human antibody which inhibit FXa generation with an IC₅₀ value lower than 100 nM (in an assay with a FVIIa concentration of 0.1 nM) In a further embodiment the IC₅₀value is lower than 10 nM. In a further embodiment the IC₅₀ value is lower than 5 nM. In a further embodiment the IC₅₀ value is lower than 1 nM . In a further embodiment the IC₅₀ value is lower than 0.1 nM.

In a further embodiment of the invention the method for preparation of a human antibody comprises testing antibodies in a FXa generation assay and selecting human antibody which inhibit FXa generation with an IC₅₀ value lower than the IC₅₀ value of FFR-rFVIIa+100 nM (using 0.1 nM FVIIa in the assay), such as lower than the IC₅₀ value of FFR-rFVIIa+10 nM, preferably lower than the IC₅₀ value of FFR-rFVIIa+5 nM, preferably lower than the IC₅₀ value of FFR-rFVIIa+1 nM, more preferably lower than the IC₅₀ value of FFR-rFVIIa+0.1 nM, more preferably lower than the IC₅₀ value of FFR-rFVIIa.

In a further embodiment of the invention the method for preparation of a human antibody comprises testing antibodies in a FVIIa/TF amidolytic assay and selecting human antibody which inhibit TF-induced FVIIa amidolytic activity with an IC₅₀ value lower than 100 nM (in an assay with a FVIIa concentration of 10 nM). In a further embodiment the IC₅₀ value is lower than 40 nM. In a further embodiment the IC₅₀ value is lower than 20 nM. In a further embodiment the IC₅₀ value is lower than 10 nM. In a further embodiment the IC₅₀ value is lower than 5 nM.

In a further embodiment of the invention the method for preparation of a human antibody comprises testing antibodies in a FVIIa/TF amidolytic assay and selecting human antibody which inhibit TF-induced FVIIa amidolytic activity, with an IC₅₀ value lower than the IC₅₀ value of FFR-rFVIIa+100 nM (using 10 nM FVIIa in the assay), such as lower than the IC₅₀ value of FFR-rFVIIa+40 nM, preferably lower than the IC₅₀ value of FFR-rFVIIa+20 nM, preferably lower than the IC₅₀ value of FFR-rFVIIa+10 nM, more preferably lower than the IC₅₀ value of FFR-rFVIIa.

In a further embodiment of the invention the method for preparation of a human antibody comprises testing antibodies in a FVIIa competition assay and selecting human antibody which compete with FVIIa binding.

In a further embodiment of the invention the method for preparation of a human antibody comprises testing antibodies in a TF ELISA assay comprising TF and selecting human antibody which bind human TF.

In a further embodiment of the invention the method for preparation of a human antibody comprises testing antibodies in a direct TF ELISA assay comprising immobilized TF and selecting human antibodies which binds immobilized human TF.

In a further embodiment of the invention the method for preparation of a human antibody comprises testing antibodies in an indirect TF ELISA assay comprising TF in solution and selecting human antibodies which binds human TF in solution

In a further embodiment of the invention the method for preparation of a human antibody comprises testing antibodies in a FXa generation assay on TF expressing cell and selecting human antibodies which inhibits FXa generation on TF expressing cell with an IC₅₀ value lower than 500 nM (in an assay with a FVIIa concentration of 1 nM). In a further embodiment the IC₅₀ value is lower than 100 nM. In a further embodiment the IC₅₀ value is lower than 50 nM In a further embodiment the IC₅₀ value is lower than 10 nM In a further embodiment the IC₅₀ value is lower than 5 nM.

In a further embodiment of the invention the method for preparation of a human antibody comprises testing antibodies in a FXa generation assay on TF expressing cell and selecting human antibodies which inhibits FXa generation on TF expressing cell with an IC₅₀ value lower than the IC₅₀ value of FFR-rFVIIa+500 nM (using 1 nM FVIIa in the assay), such as lower than the IC₅₀ value of FFR-rFVIIa+100 nM, preferably lower than the IC₅₀ value of FFR-rFVIIa+50 nM preferably lower than the IC₅₀ value of FFR-rFVIIa+10 nM, more preferably lower than the IC₅₀ value of FFR-rFVIIa+5 nM, more preferably lower than the IC₅₀ value of FFR-rFVIIa.

The term “TF expressing cell” mean any mammalian cell, that expresses human TF.

In a further embodiment of the invention the method for preparation of a human antibody comprises testing antibodies in a whole cell TF binding assay and selecting human antibodies which competes with FVIIa binding to human TF expressed on the cell surface of whole cells.

In a further embodiment of the invention the method for preparation of a human antibody comprises testing antibodies in a biosensor assay and selecting human antibodies with a K_{d} value for binding to human TF lower than 100 nM. In a further embodiment the K_{d} value for binding to human TF is lower than 10 nM. In a further embodiment the K_{d} value for binding to human TF is lower than 5 nM. In a further embodiment the K_{d} value for binding to human TF is lower than 1 nM. In a further embodiment the K_{d} value for binding to human TF is lower than 0.5 nM. In a further embodiment the K_{d} value for binding to human TF is lower than 10⁻¹⁰ M. In a further embodiment the K_{d} value for binding to human TF is lower than 10⁻¹¹ M. In a further embodiment the K_{d} value for binding to human TF is lower than 10⁻¹² M. In a further embodiment the K_{d} value for binding to human TF is lower than 10⁻¹³ M. In a further embodiment the K_{d} value for binding to human TF is lower than 10⁻¹⁴ M. In a further embodiment the K_{d} value for binding to human TF is lower than 10⁻¹⁵ M.

In a further embodiment of the invention the method for preparation of a human antibody comprises testing antibodies in a MAPK signalling assay and selecting human antibodies which inhibits FVIIa-induced activation of the MAPK signalling. In one embodiment the human antibody inhibits FVIIa-induced activation of the MAPK signalling with 98%. In one embodiment the human antibody inhibits FVIIa-induced activation of the MAPK signalling with 90%. In one embodiment the human antibody inhibits FVIIa-induced activation of the MAPK signalling with 70%. In one embodiment the human antibody inhibits FVIIa-induced activation of the MAPK signalling with 50%. In one embodiment the human antibody inhibits FVIIa-induced activation of the MAPK signalling with 30%.

The term “MAPK signalling” is intended to mean a cascade of intracellular events that mediate activation of Mitogen-Activated-Protein-Kinase (MAPK) or homologues thereof in response to various extracellular stimuli. Three distinct groups of MAP kinases have been identified in mammalian cells: 1) extracellular-regulated kinase (Erk1/2 or p44/42), 2) c-Jun N-terminal kinase (JNK) and 3) p38 kinase. The Erk1/2 pathway involves phosphorylation of Erk 1 (p 44) and/or Erk 2 (p 42). Activated MAP kinases e.g. p44/42 MAPK can translocate to the nucleus where they can phosphorylate and activate transcription factors including (Elk 1) and signal transducers and activators of transcription (Stat). Erk1/2 can also phosphorylate the kinase p90RSK in the cytoplasm or in the nucleus, and p90RSK then can activate several transcription factors.

The term “protein kinase” is intended to indicate an enzyme that is capable of phosphorylating serine and/or threonine and/or tyrosine in peptides and/or proteins.

The term “FVIIa-induced activation of the MAPK signalling” is intended to indicate that FVIIa binds to TF in a mammalian cell and thereby induce MAPK signalling.

In a further embodiment of the invention the method for preparation of a human antibody comprises testing antibodies in a gene expression analysis assay (assay 15) and selecting human antibodies which inhibits FVIIa induced up-regulation of genes independently selected from the list comprising Fra-1, Id2, and Cyr61.

It is to be understood, that antibodies against TF, which inhibits the activity of TF may bind different epitopes present on TF and may inhibit both the binding of FVIIa or the binding of FX or FXa to human TF. It is an object of the present invention to select antibodies, which inhibits the binding of FVIIa to human TF and thereby the FVIIa induced intracellular signalling.

In a further embodiment of the invention the method for preparation of a human antibody comprises testing antibodies in a human cancer assay (assay 13) and selecting human antibodies which inhibits growth or metastasis of human cancers.

In a further embodiment of the invention the isolated human antibody inhibits FVIIa induced up-regulation of genes independently selected from the list comprising Fra-1, Id2, and Cyr61.

In a further embodiment of the invention the isolated human antibody does not inhibit binding of FX or FXa to human TF.

In a further embodiment of the invention the isolated human antibody inhibits the intracellular activity of TF.

In a further embodiment of the invention the method for preparation of a human antibody comprises testing antibodies in an epitope mapping assay and selecting human antibodies which binds preferred epitopes on TF. In one embodiment the preferred epitope comprises one or more of the residues Trp45, Lys46 and Tyr94. In one embodiment the preferred epitope comprises the residue Trp45. In one embodiment the preferred epitope comprises the residue Lys46. In one embodiment the preferred epitope comprises the residue Tyr94.

In a further embodiment of the invention the isolated human antibody binds to an epitope within the interface between TF and FVIIa.

The residues in TF that are responsible for the interaction between the protease domain of FVIIa and TF determined from the X-ray structure (Banner et al. 1996 Nature, 380: 41-46) are; Ser39, Gly43, Trp45, Ser47, Phe50, Arg74, Phe76, Tyr94, Pro92. This interface between the protease domain of FVIIa and TF is characterized as being a complex interface region containing many intermolecular hydrogen bonds allowing many fine contacts between TF and FVIIa to obtain high specificity in binding process of FVIIa.

The present invention also relates to high affinity human monoclonal antibodies to TF. The TF surface containing the contact interface for the protease domain of FVIIa holds a specific topology that are prone to react to create protein-protein interactions, wherein another type of protein-protein interaction is the complex formation between an antibody and a protein ligand. Thus, monoclonal antibodies directed against this epitope on human TF gives high affinity Mabs.

One aspect of the present invention is high affinity human monoclonal antibodies, that are immunoreacting with the contact interface for the protease domain of FVIIa.

Human TF antibodies of the present invention act as antagonists for TF-mediated induction of coagulation, thus inhibiting the binding of coagulation FVIIa to TF and thereby blocking the production of thrombin and the subsequent deposition of fibrin. Human TF antibodies are particularly useful for administration to humans to treat a variety of conditions involving intravascular coagulation. As such, human TF antibodies may be useful for inhibiting TF activity resulting in, for example, the inhibition of blood coagulation, thrombosis or platelet deposition. Furthermore, human TF antibodies according to the present invention, which acts to inhibit the cellular functions of TF, the signalling function of TF, may be useful in conditions like sepsis, inflammation, atherosclerosis, restenosis, or cancer.

Human TF antibodies may be useful in a variety of diseases. Included are thrombotic or coagulopathic related diseases or disorders including inflammatory response and chronic thromboembolic diseases or disorders associated with fibrin formation including vascular disorders such as deep venous thrombosis, arterial thrombosis, post surgical thrombosis, coronary artery bypass graft (CABG), percutaneous transdermal coronary angioplastry (PTCA), stroke, tumor growth, tumor metastasis, angiogenesis, thrombolysis, arteriosclerosis and restenosis following angioplastry, acute and chronic indications such as inflammation, septic chock, septicemia, hypotension, adult respiratory distress syndrome (ARDS), systemic inflammatory response syndrome (SIRS), disseminated intravascular coagulopathy (DIC), pulmonary embolism, pathological platelet deposition, myocardial infarction, or the prophylactic treatment of mammals with atherosclerotic vessels at risk for thrombosis, venoocclusive disease following peripheral blood progenitor cell (PBPC) transplantation, hemolytic uremic syndrome (HUS), thrombotic thrombocytopenic purpura (TTP) and other diseases or disorders. The human TF antibodies may be used to prevent the occurrence of thromboembolic complications in identified high risk patients, such as those undergoing surgery or those with congestive heart failure. The human TF antibodies may be particularly useful in the treatment of intimal hyperplasia or restenosis due to acute vascular injury. Acute vascular injuries are those which occur rapidly (i.e. over hours to months, in contrast to chronic vascular injuries (e.g. atherosclerosis) which develop over a lifetime. Acute vascular injuries often result from surgical procedures such as vascular reconstruction, wherein the techniques of angioplasty, endarterectomy, atherectomy, vascular graft emplacement or the like are employed. Hyperplasia may also occur as a delayed response in response to, e.g., graft emplacement or organ transplantation. Since human TF antibodies is more selective than heparin, binding only TF which has been exposed at sites of injury, and because human TF antibodies does not destroy or inhibit other coagulation proteins, it will be more effective and less likely to cause bleeding complications than heparin when used prophylactically for the prevention of deep vein thrombosis.

As shown in the examples that follow, the human TF antibodies of the present invention is able to bind selectively to cell-surface TF and inhibit its functional activity by inhibiting the binding of coagulation FVIIa to TF. Human TF antibodies which maintain binding to TF inhibit platelet accumulation at the site of vascular injury by blocking the production of thrombin and the subsequent deposition of fibrin.

Due to the ability of human TF antibodies to block thrombin generation and limit platelet deposition at sites of acute vascular injury, human TF antibodies which maintain binding to TF thereby inhibiting FVIIa binding can be used to inhibit vascular restenosis.

Compositions comprising human TF antibodies are particularly useful in methods for treating patients when formulated into pharmaceutical compositions, where they may be given to individuals suffering from a variety of disease states to treat coagulation-related conditions. Such human TF antibodies, capable of binding TF and inhibiting FVIIa binding to TF, may possess a longer plasma half-life and thus a correspondingly longer period of anticoagulant activity when compared to other anticoagulants. Among the medical indications for the subject compositions are those commonly treated with anticoagulants, such as, for example, deep vein thrombosis, pulmonary embolism, stroke, disseminated intravascular coagulation (DIC), fibrin deposition in lungs and kidneys associated with sepsis, antiphospholipid syndrome (APS), atherosclerosis and myocardial infarction. The compositions can be used to inhibit vascular restenosis as occurs following mechanical vascular injury, such as injury caused by surgery, microsurgery, balloon angioplasty, endarterectomy, reductive atherectomy, stent placement, laser therapy or rotablation, or as occurs secondary to vascular grafts, stents, bypass grafts or organ transplants. The compositions can thus be used to inhibit platelet deposition and associated disorders. Thus, a method of inhibiting coagulation, vascular restenosis or platelet deposition, for example, comprises administering to a patient a composition comprising human TF antibodies in an amount sufficient to effectively inhibit coagulation, vascular restenosis or platelet deposition. The methods also find use in the treatment of acute closure of a coronary artery in an individual (e.g. acute myocardial infarction), which comprises administering the human TF antibodies, in conjunction with tissue plasminogen activator or streptokinase, and can accelerate tPA induced thrombolysis. The human TF antibodies are given prior to, in conjunction with, or shortly following administration of a thrombolytic agent, such as tissue plasminogen activator.

According to the invention, human monoclonal antibodies directed against human TF may be produced by immunizing transgenic mice (Obtained from Medarex) carrying parts of the human immune system rather than the mouse system with human TF. Splenocytes from these transgenic mice are used to produce hybridomas that secrete human monoclonal antibodies as described (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368: 856-859; Green, L. L. et al. 1994 Nature Genet. 7: 13-21; Morrison, S. L. et al. 1994 Proc. Natl. Acad. Sci. USA 81: 6851-6855; Bruggeman et al. 1993 Year Immunol 7: 33-40; Tuaillon et al. 1993 PNAS 90: 3720-3724; Bruggeman et al. 1991 Eur J Immunol 21: 1323-1326).

Human monoclonal antibodies directed against human TF may also be produced by phage display. Human antibody libraries can be constructed from immunized persons and displayed on the surface of filamentous phage. High-affinity human single-chain Fv (ScFv) and Fab antibody fragments have in numerous of cases been isolated from such libraries using a panning technique in which the antigen of interest is immobilised on a solid surface i.e. microtiter plates or beads (Barbas C. F., III and Burton, D. R. Trends. Biotechnol. 1996, 14:230-234; Aujame L. et al, Hum. Antibodies 1997, 8:155-68). Phage display of large naïve libraries has also made it possible to isolate human antibodies directly without immunization (DeHaard H. J. et al J. Biol. Chem. 1999, 18218-18230).

### BRIEF DESCRIPTION OF THE DRAWINGS

The present invention is described in further detail in the examples with reference to the appended drawings wherein

FIG. 1. Schematic presentation of an examplified screening assay for selection of human monoclonal high affinity antibodies against TF.

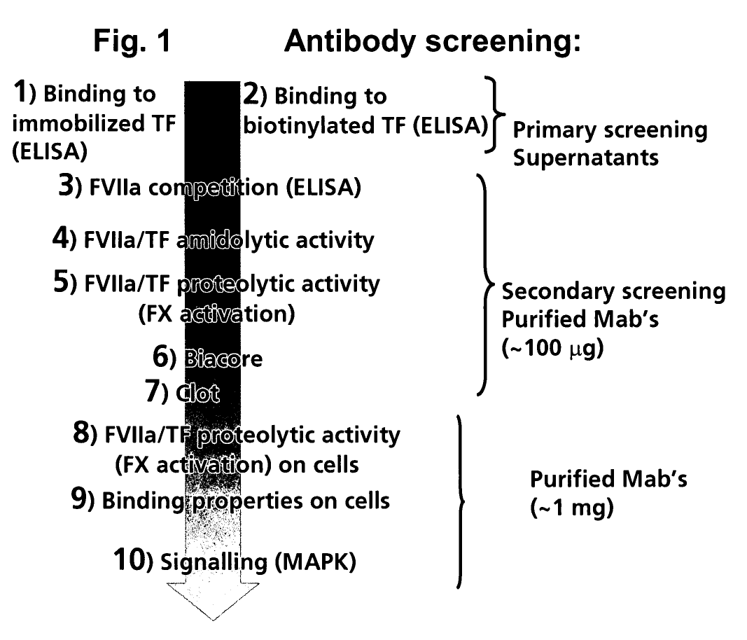

FIG. 2. Detailed schematic representation of screening assays 1-3 as described in example 1.

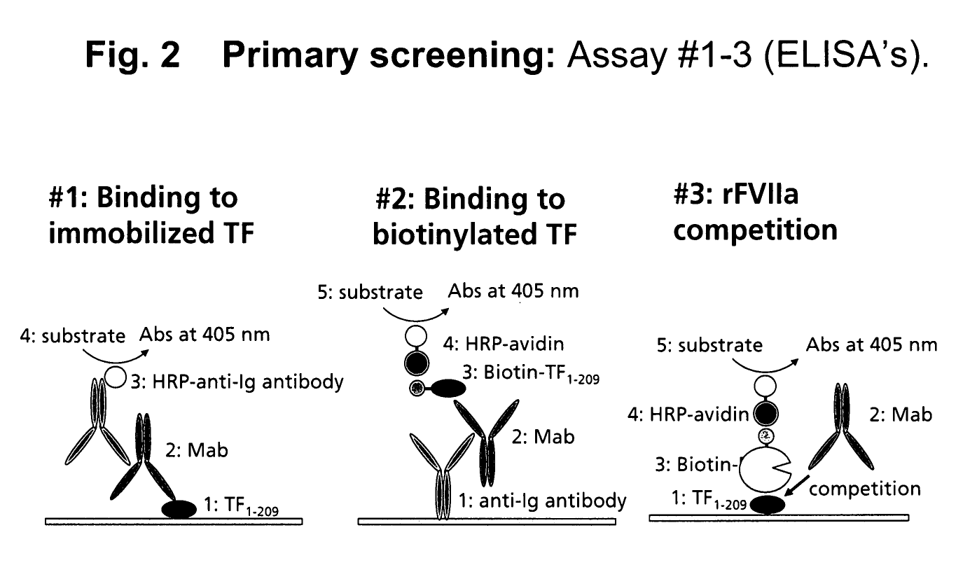

FIG. 3. Detailed schematic representation of screening assays 4-7 as described in example 1.

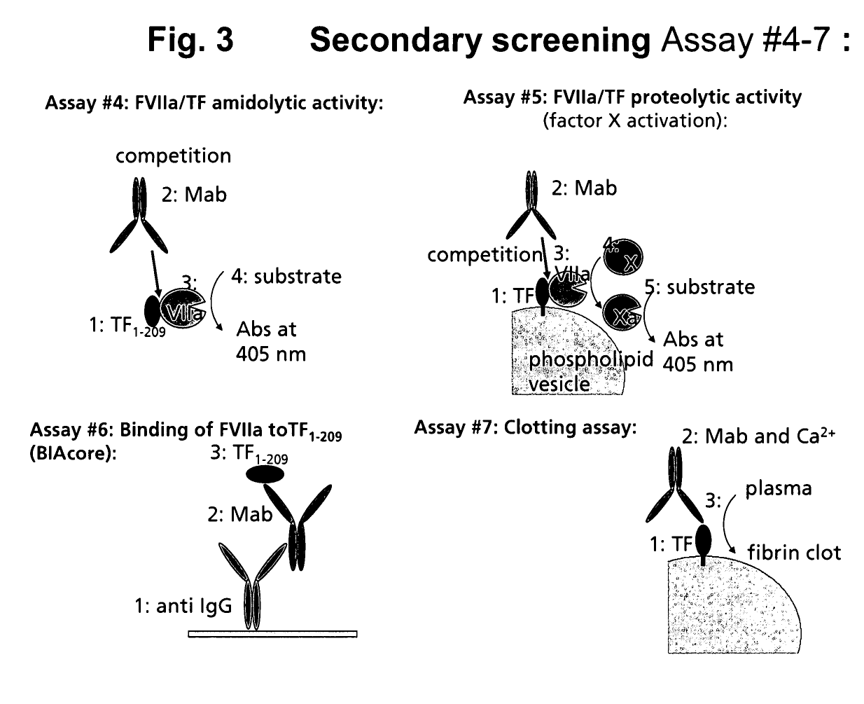

FIG. 4. Detailed schematic representation of screening assays 8-10 as described in example 1.

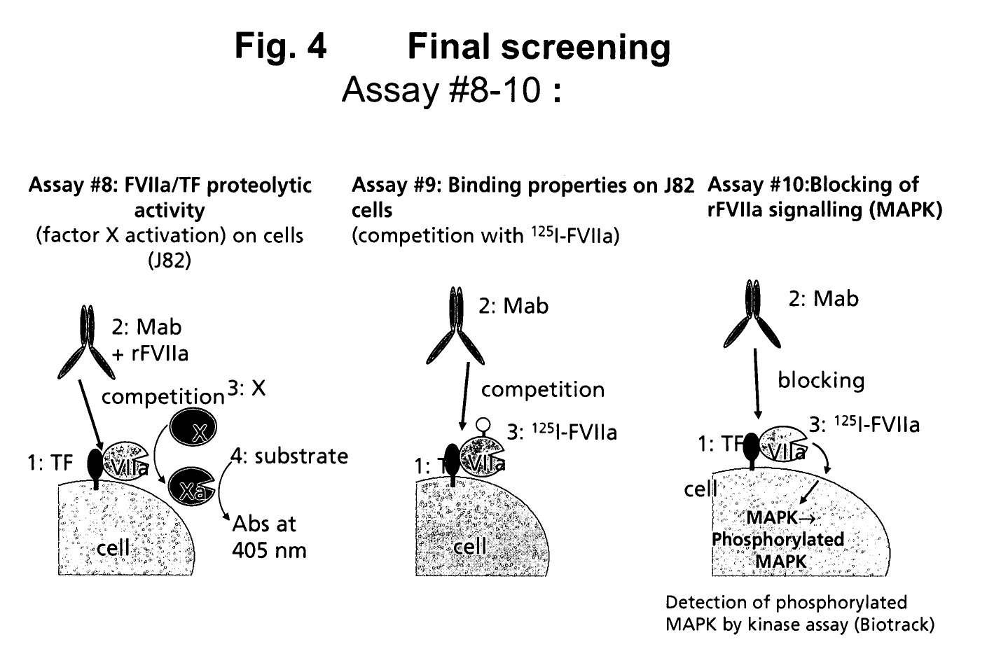

FIG. 5. An example of screening antibodies by assay no. 4. Inhibition of sTF/FVIIa amidolytic activity by FFR-rFVIIa (closed circles) and the human anti TF monoclonal antibody HuTF-31F2 (open circles).

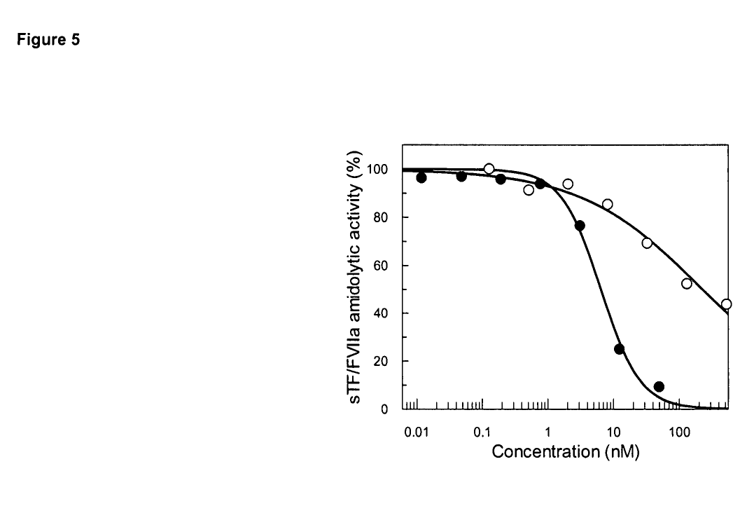

FIG. 6. An example of screening antibodies by assay no. 5. Inhibition of factor Xa generation by FFR-rFVIIa (closed circles) and the human anti-TF monoclonal antibody HuTF-31F2 (open circles).

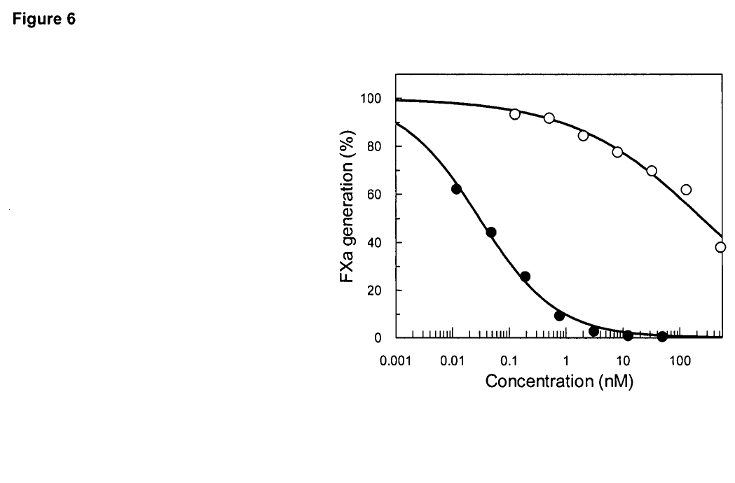

FIG. 7. An example of screening antibodies by assay no. 7. Inhibition of human TF-induced clotting by FFR-rFVIIa (closed circles) and the human anti TF monoclonal antibody HuTF-31F2(open circles).

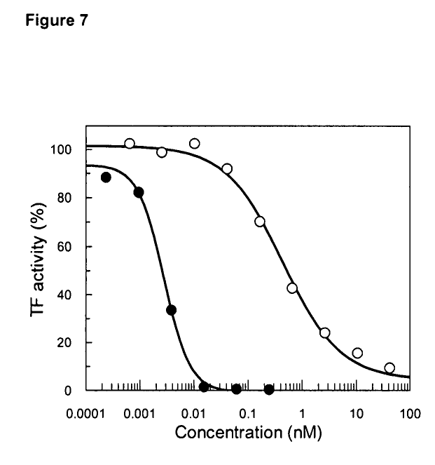

FIG. 8. An example of screening antibodies by assay no. 10. Only anti-TF monoclonal antibodies preventing FVIIa binding inhibits TF/FVIIa-mediated signaling.

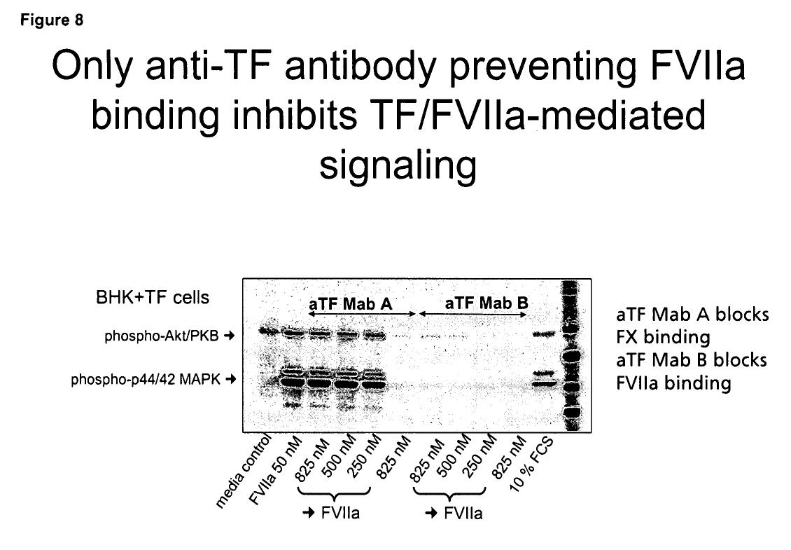

FIG. 9. Human anti TF Mab inhibit FVIIa induced phosphorylation of p44/42 MAPK (assay no. 10). BHK cell transfected with TF were serum-starved for 2 hr to make cells quiescent. The antibodies HuMab 30F5 (500 nM) and HuMab 31F2 (500 nM) were added to the cells 15 min prior addition of FVIIa (15 nM). Cells were lysed and proteins were separate on SDS-PAGE and transferred to nitrocellulose by electroblotting. Western blot analysis was performed using polyclonal phospho-specific antibodies to p44/42 MAPK. Secondary antibodies were anti-rabbit IgG conjugated to Horse Radish Peroxidase. Detection of chemiluminescence was performed using a cooled CCD-camera. The bands on the digitalized picture were quantified and the band obtained with FVIIa was set to 100%. When cells were pre-incubated with HuMab 30F5 (500 nM) a 50% reduction in the phosphorylated band was observed and when cells were pre-incubated with HuMab 31F2 (500 nM) a 25% reduction was observed. In conclusion, this experiment show that the human antibodies against TF (30F5 and 31F2) partially inhibited the FVIIa induced phosphorylation of p44/42 MAPK. Similar results were obtained using 50 nM FVIIa.

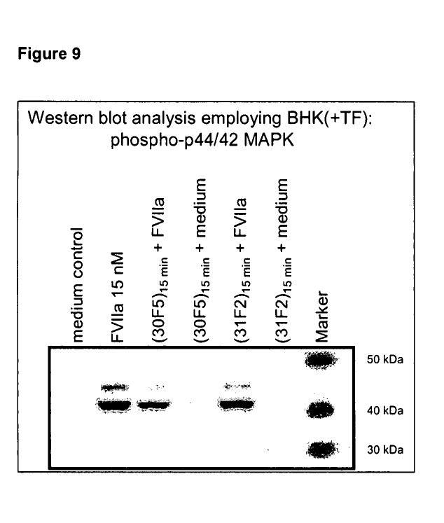

FIG. 10. An example of screening antibodies by assay no. 16. The figure demonstrates the inhibition of TF intracellular activity in TF expressing cells by monoclonal antibodies against TF. Anti TF Mab B inhibits TF intracellular activity, while Anti-TF Mab A do not.

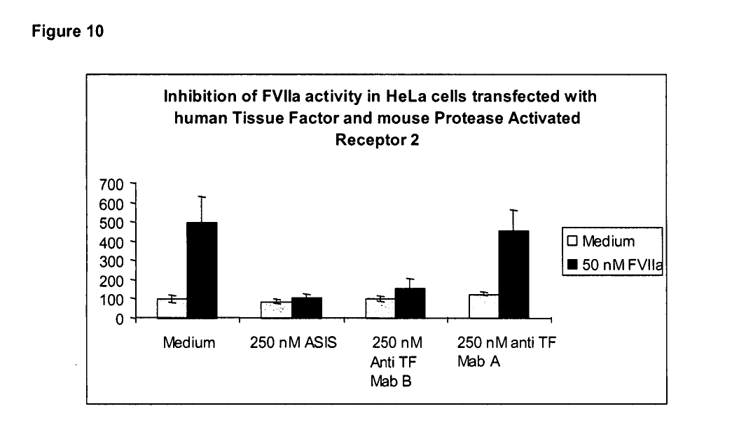

FIG. 11. An example of screening antibodies by assay no. 12. Velocity profile of thromboelastograms obtained with 0.5 nM of FFR-rFVIIa and the human anti-TF antibody HuTF-31F2.

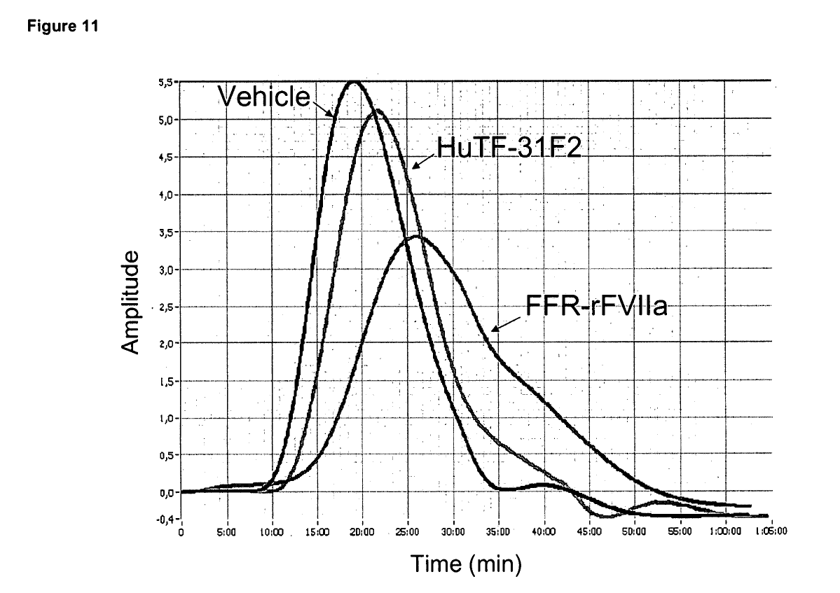

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### Example 1

### Preparation of Human Mabs Immunospecific for Human TF

Reagents.

Human TF can be isolated from human brain as described by Rao, L. V. M., Thrombosis Research, 51:373-384 1988.

Lipidated recombinant human TF (Dade Innovin, Baxter) can also be used as human thromboplastin reagent. Rat, rabbit, baboon, and pig thromboplastin are prepared from brain tissue. Two volumes of 45° C. 0.9% NaCl are added to the brain tissue, and the tissue is homogenized with a manual glass homogenisator. After 30 min incubation at 45° C. with occasional shaking, the samples are centrifuged 20 min at 2000×g. The precipitate are discarded, and the supernatant is aliquoted and stored at −80° C. until use.

Relipidated TF may be obtained by reconstitution of recombinant human full length TF (American Diagnostica #4500) into phospholipid vesicles (PC/PS 75/25).

Biotinylated human TF is produced as follows: Biotin-NHS (n-succinimido biotin, Sigma H-1759) is dissolved in DMF (dimethylformamid) at a concentration of 1.7 mg/ml. 1 mg/ml of human TF in 0.1 M NaHCO₃ buffer is added 60 μl of the biotin-NHS solution and is allowed to react for 4 hours at room temperature. The reacting solution containing the biotinylated TF is dialysed against PBS-buffer over night.
FVIIa used is recombinant human FVIIa prepared as described by Thim, L. et al. Biochem 27: 7785-7793, 1988.
sTF: Recombinant human soluble TF₁₋₂₀₉ expressed in E. coli and purified essentially as described by Freskg\dot{a}rd, P.-O. et al. Protein Sci. 5, 1531-1540 (1996).
S2288: reconstituted in H₂O to 17.24 mg/ml (Chromogenix)
FX: Purified human plasma FX (HFX, Enzyme Research Laboratories Ltd.)
FXa: Purified human plasma FX activated with Russel's Viper Venom (HFXa, Enzyme Research Laboratories Ltd.)
Chromozyme X: Dissolved in H₂O to 1.25 mg/ml. (Boehringer Mannheim)
¹²⁵I-FVIIa is obtained by standard radiolabelling procedures.
FFR-rFVIIa: FVIIa blocked in the active site with D-Phe-L-Phe-L-Arg-chloromethyl ketone.
   Prepared as described by Sorensen B. B. et al. J.Biol.Chem. 272: 11863-11868, 1997.
   Immunization.

Human TF is emulsified in Freunds Complete Adjuvant. HuMab mice or hybrids therof (Medarex) are given 40 μg by a subcutaneous injection. After 14 and 28 days, and eventually more times with intervals of 14 days the mice are boosted with a similar injection of 20 μg of TF in Incomplete Freunds Adjuvant. Ten days after the last injection a blood sample is taken and sera are tested for human TF specific antibodies by TF ELISA (Assay 1 and 2).

Fusion.

Mice with positive serum test from assay 1-3 are boosted with 20 μg of human TF by an intravenous injection and sacrificed after three days. The spleen is removed aseptically and dispersed to a single cell suspension.

Fox-myeloma cells are grown in CD Hybridoma medium (Gibco 11279-023).

Fusion of spleen cells and myeloma cells (P3×63 Ag8.653, ATCC CRL-1580), and the Sp2/0 (ATCC CRL-1581) myeloma cell lines for our fusions are done by the PEG-methods (Köhler, G & Milstein C. (1976), European J. Immunology, 6:511-19). Cells are seeded in microtiter plates and incubated at 37° C. Medium is changed three times over the next two weeks. 100 μl of supernatant from hybridoma cells is removed from each well and tested for TF specific antibodies in TF ELISA (Assay 1 and 2).

### Example 2

Screening.

The various assays used in the screening of serum and culture supernatants for specific selected antibodies are described in the following:

Direct TF ELISA Assay (Assay 1):

Nunc immunoplates are coated overnight at 4° C. with 1 μg/ml of human sTF in PBS. Plates are blocked with blocking buffer (TBS with 5 mM CaCl₂ and 2% BSA) and washed in TBS+0.05% Tween 20, and the supernatants from the hybridoma cells are added. After incubation at room temperature for 1 hour, plates are washed and anti-human IgG labelled with horseradish peroxidase (HRPO) is added. After another hour of incubation, plates are washed and developed with TMB-substate (Kem-EN-Tec) as described by the manufactures. Absorbance at 450 nm is measured on an ELISA-reader.

Indirect TF ELISA Assay (Assay 2):

Nunc-immunoplates are coated with 0.5 μl/ml of goat anti-human IgG (Southern Biotechnology Associates, Cat-#2040-1) in PBS and incubated overnight at 4° C. Plates are blocked with blocking buffer (TBS with 5 mM CaCl₂ and 2% BSA) and washed in TBS+5 mM CaCl₂+0.05% Tween 20. Culture supernatants from the hybridoma cells are added and the plates incubated for 1 hour at room temperature. After another wash, biotinylated human sTF are added at a concentration of 1 pg/ml, and incubated for 1 hour. After washing, 100 μl of a Streptavidin-HRPO solution is added and incubated for 1 hour. Plates are developed with TMB-substate as described for assay 1.

FVIIa Competition Assay (Assay 3):

Nunc-immunoplates are incubated with human sTF (conc 5 μg/ml in PBS) over night, 4° C. Plates are washed and blocked in TBS buffer with 5 mM CaCl₂ and 2% BSA. Anti-human TF Mabs are added and plates are incubation for 2 hours. Plates are washed before biotinylated human FVIIa are added (1 μg/ml in TBS buffer with 5 mM CaCl₂ and 2% BSA) and the plates incubated for 1 hour. Plates are washed before addition of HRPO-labeled Streptavidin and incubated for 45 min. Plates are washed again before development with TMB substrate (Kem-EN-Tec) as described by the manufactures.

Inhibition of FVIIa/sTF Amidolytic Activity (Assay 4):

Inhibition of FVIIa-TF catalyzed amidolytic activity by anti-human TF Mabs is tested employing soluble human TF (10 nM), recombinant human FVIIa (10 nM) and increasing concentrations of Mabs (0.0122-50 nM). Varying concentrations of anti-human TF Mabs or FFR-rFVIIa are preincubated with 10 nM sTF and 10 nM FVIIa in BSA buffer (50 mM Hepes, pH 7.4, 100 mM NaCl, 5 mM CaCl₂ and 1 mg/ml BSA) for 60 min at room temperature before addition of substrate S2288 (1.2 mM, Chromogenix). The colour development is measured continuously for 30 min at 405 nm. Amidolytic activity is presented as mOD/min. IC₅₀ values for inhibition of FVIIa/TF amidolytic activity by the Mabs may be calculated. The IC₅₀ value for FFR-rFVIIa is 7+/−3 nM in this assay.

Inhibition of FXa Generation (Assay 5).

Lipidated TF (10 pM), FVIIa (100 pM) and anti-TF Mabs or FFR-rFVIIa (0-50 nM) in BSA buffer (see assay 4) are incubated 60 min at room temperature before FX (50 nM ) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC₅₀ values for Mab inhibition of FVIIa/lipidated TF-mediated activation of FX may be calculated. The IC₅₀ value for FFR-rFVIIa is 51+/−26 pM in this assay.

Biosensor Assay (Assay 6):

Antibodies are tested on the Biacore instrument by passing a standard solution of anti-human TF Mab over a chip with immobilized antibody to human IgG. This is followed by different concentrations of sTF in 10 mM hepes pH 7.4 containing 150 mM NaCl, 10 mM CaCl₂ and 0.0003% polysorbate 20. K_{d} values are calculated from the sensorgrams using the integrated Biacore evaluation software.

TF-Dependent Clotting Assay (Assay 7):

The assay is carried out on an ACL300 Research clotting apparatus (ILS Laboratories). Dilutions of anti-human TF Mabs in 50 mM imidazole, pH 7.4, 100 mM NaCl, 0.1% BSA are mixed with 25 mM CaCl₂ in the ratio of 2 to 5 and added to sample cups in the clotting apparatus. Thromboplastin from human, rat, rabbit, baboon, or pig diluted with the imidazole buffer to give clotting time of approximately 30 sec in samples without antibody is placed in reagent reservoir 2, and human, rat, rabbit, baboon, or pig plasma, in reagent reservoir 3. During the analysis 70 μl of the antibody and CaCl₂ mixture is transferred to 25 μl thromboplastin reagent and preincubated 900 sec before addition of 60 μl plasma and measuring of the clotting time. Maximal clotting time is set to 400 sec. A dilution of the thromboplastin is used as standard curve for converting clotting times into TF activity relative to the control without anti-TF Mabs or FFR-rFVIIa added. The IC₅₀ value for FFR-rFVIIa is 4.4+/−0.4 pM in this assay.

Inhibition of FVIIa/Cell Surface TF Catalyzed Activation of FX by Mabs (Assay 8):

Monolayers of cells expressing human TF, e.g. human lung fibroblasts WI-38 (ATTC No. CCL-75), human bladder carcinoma cell line J82 (ATTC No. HTB-1), human keratinocyte cell line CCD 1102KerTr (ATCC no. CRL-2310), human glioblastoma cell line U87, or human breast cancer cell line MDA-MB231, are employed as TF source in FVIIa/TF catalyzed activation of FX. Confluent cell monolayers in a 24-, 48- or 96-well plate are washed one time in buffer A (10 mM Hepes, pH 7.45, 150 mM NaCl, 4 mM KCl, and 11 mM glucose) and one time in buffer B (buffer A supplemented with 1 mg/ml BSA and 5 mM Ca²⁺). FVIIa (1 nM), FX (135 nM) and varying concentrations of Mab (or FFR-rFVIIa) in buffer B are simultaneously added to the cells. Alternatively the cells are preincubated 15 min with anti-TF Mabs or FFR-rFVIIa before addition of rFVIIa and FX. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of colour development are converted to FXa concentrations using a FXa standard curve. The IC₅₀ value for FFR-rFVIIa is 1.5 nM in this assay.

Inhibition of ¹²⁵I-FVIIa Binding to Cell Surface TF by Mab (Assay 9):

Binding studies are employed using cells expressing human TF, e.g. human lung fibroblasts WI-38 (ATTC No. CCL-75), human bladder carcinoma cell line J82 (ATTC No. HTB-1), human keratinocyte cell line CCD 1102KerTr (ATCC no. CRL-2310), human glioblastoma cell line U87, or human breast cancer cell line MDA-MB231. Confluent monolayers in 24-well tissue culture plates are washed once with buffer A (see assay 8) and once with buffer B (see assay 8). The monolayers are preincubated 2 min with 100 μl cold buffer B. Varying concentrations of Mabs (or FFR-rFVIIa) and radiolabelled FVIIa (0.5 nM ¹²⁵I-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC₅₀ value for FFR-rFVIIa is 4 nM in this assay.

Inhibition of FVIIa/TF-Induced p44/42 MAPK Activation by Mab (Assay 10):

The amount of phosphorylated p44/42 MAPK and/or Akt, and/or p90RSK is determined by quantitative detection of chemiluminescence (Fujifilm LAS-1000) from western blot analysis. Cells expressing human TF, e.g. CCD1102KerTr, NHEK P166, human glioblastoma cell line U87, or human breast cancer cell line MDA-MB231, are cultured in medium with 0-0.1% FCS for 24 or 48 hours prior to the experiment to make cells quiescent. At the day of the experiment the cells must be 70-80% confluent. The experiment is performed by preincubating the cells with excess Mab or FFR-rFVIIa in medium without serum for 30 min at 37° C. before addition of 10-100 nM FVIIa and incubating for 10 min. As a positive control of cell signaling, cells are treated with 10% FCS for 10 minutes. Cells are washed 2 times in ice-cold PBS before cells are lysed in lysis buffer (20 mM Tris, 0.1% Triton X-100, 1 mM EDTA, 1 mM EGTA, 50 mM sodium-fluoride, 10 mM sodium β-glycerophosphate, 5 mM sodium pyrophosphate, 150 mM NaCl, pH 7.5 containing 0.1 mM 4-(2-aminoethyl)benzenesulfonyl fluoride (AEBSF) and 1 mM benzamidine. Added just before use: 1 mM sodium orthovanadate, 5 μg/ml leupeptin, 10 μg/ml aprotinin). Lysates were mixed with SDS-sample buffer and loaded on a SDS-polyacrylamide gel. A standard biotinylated protein marker is loaded on each gel. Proteins separated on the SDS-polyacrylamide gel were transferred to nitrocellulose by electroblotting, and the kinases p44/42 MAPK, Akt and p90RSK were visualized by immunoblotting with phosphospecific antibodies, and chemiluminiscence is quanitiated by Fujifilm LAS1000.

Epitope Mapping Assay (Assay 11):

Preparation of Soluble TF (sTF) Variants.

sTF variants (I22C, W45C, K46C, Y94C, F140C, W158C, K201C) are constructed using inverse PCR (QuikChange, Stratagene, La Jolla, Calif., USA) using the wild type plasmid (Freskg\dot{a}rd et al. Protein Sci. 5, 1531-1540, 1996) as template. The wild type and variants are expressed and purified in E. coli as described elsewhere (Freskgard et al., Protein Sci. 5, 1531-1540, 1996) with some modifications. The cell lysis is performed by the X-press (Biox, Sweden) technique in 10 mM Tris-HCl buffer, pH 7.5 and thereafter resuspended in the same buffer with the addition of 1 mg of DNAse. The solution is centrifuged at 11000×g for 20 min at 4° C., and the inclusion bodies are denatured in 75 ml of 6 M GuHCl, 0.5 M NaCl, 20 mM Tris-HCl, pH 8.0. Refolding is achieved after 1-hour incubation at room temperature by dropwise diluting the denatured protein into a 1 L solution containing 50 mM Tris-HCl, 0.25 M NaCl, pH 8.0 with gentle stirring for approximately 2 hours. Purification is performed using Q-Sepharose Fast Flow (Pharmacia, Uppsala, Sweden) and FVIIa affinity chromatography as described by Freskgard et al. (1996). The homogeneity of the protein is verified by SDS-PAGE. The concentration is measured at A₂₈₀ₙₘ and determined using a calculated extinction coefficient of 37440 M⁻¹ cm⁻¹ (Gill and von Hippel, 1989).

MaxiSorp plates (Nunc-Immuno) are coated with wild type sTF and the variants (10 μg/ml) in TBS and blocked with blocking buffer (TBS with 0.1% Tween 20 and 0.5% BSA). The plates are washed with washing buffer (TBS and 0.1% Tween 20). The anti-human TF Mabs are applied at a concentration of 1 ng/ml in blocking buffer and incubated for one hour. The plates are then washed (6×) using the washing buffer. The antibody binding is subsequently detected using an HRP-labelled anti-human IgG (Helica Biosystems, Inc) at a 1:2000 dilution in blocking buffer using the TMBₚₗᵤₛ-substrate (Kem Tech Cat. 4390A). The final ELISA signal (OD₄₅₀₋₆₂₀) is used as a measure of the binding of each antibody to all sTF variants.

Thromboelastography (Assay 12)

Human thromboplastin (e.g. Innovin, Dade Behring, final dilution 50,000×) is mixed with CaCl₂ (final concentration 16.7 mM) and anti-TF Mabs and incubated 15 min at room temperature. Citrate-stabilized human whole blood (280 μl) is added to RoTEG sample cups (Pentapharm) and preheated 5 min at 37° C., before addition of 40 μl thromboplastin/CaCl₂/anti-TF Mab mixture. Thromboelastography is followed for one hour in a RoTEG apparatus (Pentapharm). Velocity profiles are obtained from the thrombograms using CoagPro Software™ (MedScience, Arhus, Denmark).

### Example 3

Human Cancer Assay. Investigating the Effects of Treatment with Human Anti-TF Mabs on Growth and Metastasis of Human Cancers in Mouse Models (Assay 13)

Treatment:

Human anti-TF Mabs given by bolus injection i.v.; 10 mg/kg=0.1 mg/10 g; Injection-volume is 0.1 ml per 10 g mouse of either of three treatment solutions:

A. Vehicle control

B. 1 mg/ml Human FFR-rFVIIa

C. 1 mg/ml anti-TF Mabs

Description of Models:

I. Primary Growth and Liver Metastasis of Colon Cancer

Healthy female athymic mice (nu/nu) aged 7-8 weeks are used. To destroy the residual immunoresistance of the nude mice to the human cell implantation, the mice are routinely irradiated at 5 Gy 2 days before human tumor grafting (Vogel et al., 1997). Mice are challenged by tumor grafting of LS174T humancolon carcinoma cells (ATCC CCL 188) cultured in RPMI 1640 with 15% fetal calf serum (FCS) as described (Li et al., Human Gene Therapy 10: 3045-3053, 1999). In brief, the cells are harvested with trypsin-EDTA, washed twice, and resuspended in serumfree RPMI supplemented with sodium-heparinate solution (1 U/ml). A small left subcostal incision is then carried out in mice under anesthesia and 3 3 106 LS174T cells in 50 ml of phosphate-buffer ed saline (PBS) are injected into the spleen. After 3 to 5 min, the spleen vessels are ligated and the spleen is surgically removed. This procedure will lead to a stable incidence of liver metastasis (more than 95%). The treatment with anti-TF Mabs will be initiated immediately after implantation and will last for the remaining study period. On days 15 and 30 after tumor cell inoculation mice are sacrificed, the livers ae removed and weighed, and the number of visible tumor nodules on the liver surfaces are counted. Liver samples are fixed overnight in AFA (5% acetic acid, 75% ethyl alcohol, 2% formalin, 18% water), transferred to 100% ethanol, and embedded in paraffin, and 5-m m sections are prepared for histological quantification of metastatic nodules, for immunohistochemistry and apoptosis quantification.

Study I-1:

| | |
|---|---|
| Aim: | To examine the effect on macroscopical growth and |
| | liver metastasis of LS174T colon tumors in nude mice of |
| | anti-TF Mabs given bolus injection i.v.; 10 mg/kg. |
| Mice: | 60 homozygotous nu/nu 6 weeks old NMRI males. |
| Groups | Mice are randomly allocated in four groups of 15 and |
| | treated with solutions A, B, or C. |
| Termination: | At a weight loss of >20% or other objective signs of severe |
| | toxicity the animal is terminated. |
| Parameters: | Tumor size in two orthogonal diameters is recorded daily |
| | during the growth phase. The body weight is recorded |
| | initially and 2-3 times per week. Postmortem |
| | determination of metastasis formation in the liver. |

II. Primary Growth and Lung Metastasis of Mammary Cancer

Human breast carcinoma cells MDA-MB-231 (ATCC HTB26) are cultured in Dulbecco's modified Eagle's medium (DMEM) supplemented with 10% fetal calf serum (FCS). MDA-MB-231 cells (3 3 106) are injected subcutaneously in nude mice (7- to 8-week-old female mice). Primary tumor growth and metastasis is evaluated as described previously (Li et al., Human Gene Therapy 12: 515-526, 2001)

Study II-1:

| | |
|---|---|
| Aim: | To examine the effect on macroscopical growth and lung |
| | metastasis of MDA-MB-231 mammary tumors in nude |
| | mice of anti-TF Mabs given bolus injection i.v.; 10 mg/kg. |
| Mice: | 60 homozygotous nu/nu 6 weeks old NMRI males. |
| Groups: | Mice are randomly allocated in four groups of 15 and |
| | treated with solutions A, B, or C. |
| Termination: | At a weight loss of >20% or other objective signs of |
| | severe toxicity the animal is terminated. |
| Parameters: | Tumor size in two orthogonal diameters is recorded daily |
| | during the growth phase. The body weight is recorded |
| | initially and 2-3 times per week. Postmortem |
| | determination of metastasis formation in the lung. |

III. Primary Growth of Glioma Tumor Xenografts

The tumor cell line MG U373 is a human glioblastoma multiforme cell line, with high angiogenic activity, high vascular density and fast growth in nude mice. Tumors are inoculated in the flanks, following standard procedures (see enclosed protocols for experimental plan). The mice are observed twice daily for signs of toxicity and the tumors are measured daily in two perpendicular diameters.

Tumors are transplanted to the flanks of nu/nu homozygotous nude mice of NMRI background. The mice are 7-week-old males obtained from M&B (Ry, Denmark). Animals are kept in a gnotobiotic environment and they receives sterile food pellets and drinking water ad libitum.

Three Different Studies is Conducted with the Glioma Tumor Model:

Study III-1:

| | |
|---|---|
| Aim: | To examine the effect on macroscopical growth of |
| | U373 tumors in nude mice of anti-TF Mabs given bolus |
| | injection i.v.; 10 mg/kg. |
| Mice: | 60 homozygotous nu/nu 6 weeks old NMRI males. |
| Groups: | Mice are randomly allocated in four groups of 15 and |
| | treated with solutions A, B, or C. |
| Termination: | At a weight loss of >20% or other objective signs of |
| | severe toxicity the animal is terminated. |
| Parameters: | Tumor size in two orthogonal diameters is recorded |
| | daily during the growth phase. The body weight is |
| | recorded initially and 2-3 times per week. |

Study III-2:

| | |
|---|---|
| Aim: | To examine the effect on macroscopic growth of U373 tumors in nude mice of |
| | anti-TF Mabs given bolus injection i.v.; 10 mg/kg. after pretherapeutic tumor |
| | growth has been established. |
| Mice: | 60 homozygotous nu/nu 6 weeks old NMRI males. |
| Groups: | Mice are randomly allocated in four groups of 15 and treated with solutions A, |
| | B, or C. Treatment starts when 6 consecutive (daily) measurements show |
| | Gompertzian growth. This corresponds to 100-200 mm³ |
| Termination: | Treatment lasts until the tumors have grown beyond the maximal size of approximately |
| | 1.0 cm³, i.e. no tumor diameter larger than 15 mm or until |
| | Gompertzian regrowth has been established by 6 consecutive measurements. |
| | At time of termination tumors from each group are excised for histological and |
| | immunochemical evaluation. At a weight loss of >20% or other objective |
| | signs of severe toxicity the animal is terminated |
| Parameters: | Tumor size in two orthogonal diameters is recorded daily during the growth |
| | phase. The body weight is recorded initially and 2 times per week. |

Study III-3:

| | |
|---|---|
| Aim: | To examine the effect of anti-TF Mabs on growth of |
| | intracranial U373 tumors in nude mice. |
| Mice: | 60 homozygotous nu/nu 6 weeks old NMRI males. |
| Tumor: | U373 implanted orthotopically in the right hemisphere |
| | following standard procedures. |
| Groups: | Mice are randomly allocated in four groups of 15 and |
| | treated with solutions A, B, or C. |
| Termination: | Mice with signs of chronic neurological |
| | impairment are euthanized. |
| Data: | Survival (i.e. time to neurological impairment) is |
| | quantified by Kaplan-Meyer statistics. |

### Example 4

(Assay 14).

In mouse wherein the TF gene is knocked out and human TF gene is inserted (mTF-KO/hTF-KI mice) a 0.5 ml matrigel plug will be located subcutaneously under the abdominal skin. In the matrigel b-FGF (5 ng) will be incorporated and one week later the formation of new patent vessels in the gel will be quantitated by measuring the content of haemoglobin (angiogenesis). The inhibitory capacity (% inhibition of the haemoglobin content) of the human anti-TF Mabs can be evaluated after single or repeated parenteral administrations of the proteins.

### Example 5

(Assay 15).

Gene Expression Analysis Assay for Discriminating Antibodies, that Prevents FVIIa Binding to TF and Antibodies, that Prevents FX Binding to TF.

In cDNA microarray analyses a specific up-regulation of three genes in BHK-TF cells treated with FVIIa has been observed. These include: Fra-1, a gene coding for Fos related antigen 1, Id2, a gene encoding a member of the helix-loop-helix class of proteins, and Cyr61 encoding an extracellular matrix signalling protein. The following assay is designed to screen for anti-TF Mabs which prevents FVIIa induced up-regulation of Fra-1, Id2 or Cyr61.

Cell Culture.

Reagents are Purchased from GIBCO-BRL Life Technologies Unless Otherwise Noted.

BHK-TF cells, created as described by Poulsen L. K. et al., J Biol. Chem. 273, 6228-6232, 1998, are grown in Dulbecco's modified Eagle's medium containing 10% FCS, 100 lU/ml penicillin, and 100 μg/ml streptomycin to obtain 95-100% confluence, washed and grown for additional 16-18 hs in medium without FCS. The cells are again washed and exposed to FCS-free medium containing 100 nM FVIIa.

For cloning of fragments for Northern blot analyses the cells are treated as follows. BHK-TF cells are grown in Dulbecco's modified Eagle's medium containing 10% FCS, 100 lU/ml penicillin, and 100 μg/ml streptomycin to obtain 95-100% confluence, washed and grown for additional 16-18 hs in medium without FCS. The cells are again washed and exposed to FCS-free medium containing 100 nM FVIIa for 1 h. CRL2091 cells (ATCC) are grown in Iscove's modified Dulbecco's medium containing 10% FBS, 100 U/ml penicillin, and 100 μg/ml streptomycin to 95-100% confluence. Subsequently, the cells are serum-starved for 16-18 hs and treated with FBS-free medium containing 100 nM FVIIa for 6 hs. Murine 3T3-L1 cells (ATCC) are maintained in Dulbecco's modified Eagle's medium supplemented with 10% fetal bovine serum, 100 U/ml penicillin, and 100 μg/ml streptomycin. Cells are grown to confluence and induced to with media containing 1 μM dexamethasone (Sigma), 10 μg/ml human insulin (Novo Nordisk A/S), and 1 μM BRL49653 (Novo Nordisk A/S) for 1 h.

Cloning of Fragments for Northern Blot Analyses.

Fra-1 is cloned by reverse transcription PCR from RNA isolated from 3T3-L1 cells treated for 1 h with dexamethasone, insulin, and BRL49653 using the superscript II kit (Life Technologies) according to the manufacturer's instructions. Id2 and Cyr61 are cloned by reverse transcription PCR from RNA isolated from BHK-TF cells treated for 1 h with FVIIa and from CRL2091 cells treated for 6 hours with FVIIa, respectively. The upstream and downstream primers are: 5′-GCGGCCGCCATGTACCGAGACTACGGGGAACCG-3′ and 5′-GCGGCCGCTCACAAAGCCAGGAGTGTAGG-3′ for Fra-1, 5′-CAGCATGAAAGCCTTCAGTC-3′ and 5′-CTCTGGTGATGCAGGCTGAC-3′ for Id2, 5′-CGTCACCCTTCTCCACTTGA-3′ and 5′-CTTGGTCTTGCTGCATTTCT-3′ for Cyr61. Parameters for PCR are one cycle of denaturing at 94° C. for 10 s, annealing at 65° C. for 15 s, and extension at 72° C. for 1.5 min, one cycle of denaturing at 94° C. for 10 s, annealing at 64° C. for 15 s, and extension at 72° C. for 1.5 min, one cycle of denaturing at 94° C. for 10 s, annealing at 63° C. for 15 s, and extension at 72° C. for 1.5 min, one cycle of denaturing at 94° C. for 10 s, annealing at 62° C. for 15 s, and extension at 72° C. for 1.5 min, one cycle of denaturing at 94° C. for 10 s, annealing at 61° C. for 15 s, and extension at 72° C. for 1.5 min, one cycle of denaturing at 94° C. for 10 s, annealing at 60° C. for 15 s, and extension at 72° C. for 1.5 min, 40 cycles of denaturing at 94° C. for 10 s, annealing at 55° C. for 15 s, and extension at 72° C. for 1.5 min. All fragments are cloned into into TOPO 2.1 (Invitrogen) and sequenced using a Megabase sequencer.

Northern Blot Analysis.

Total RNA are isolated from BHK-TF cells incubated with FVIIa, FX, ASIS, 1F44A1 or TF8-5G9 using TriZol following the instructions of the vendor. 20 μg of RNA are size-fractionated in a denaturing gel containing 1% agarose, 20 mM MOPS, 5 mM NaOAc, 6% formaldehyde, and 1 mM EDTA, transferred to a Hybond N⁺ membrane (Amersham) by capillary blotting and immobilized by UV crosslinking. cDNA encoding Fra-1, Id2 or Cyr61 are labelled with the Prime It kit (Stratagene) using [α-³²P] dATP (Amersham) and hybridized using Express Hyb (Clontech) following the manufacturer's instructions and results are visualized by autoradiography.

### Example 6

(Assay 16).

MAPK Assay via the Elk1 Transcription Factor/Luciferase Reporter (PathDetect)

HeLa cells are seeded to 40% confluence in a T-80 flask one day prior to transfection. Cells are transfected with 150 ng pFA-Elk1 (Stratagene), 3 μg pFR-Luc (Stratagene), 3 μg human TF/pcDNA3 and 3 μg mouse Protease Activated Receptor 2/pcDNA3,1+ using 36 μl FuGene (Roche) as described in the manual. The following day the cells are detached by Versene™ (Invitrogen) and seeded in black 96 well view plates (Packard) at a cell density of 20.000 cells per well. After the cells had reattached to the plate, the medium is replaced with 160 μl per well serum-free Dulbeccos Modified Eagle Medium (Invitrogen) and incubated for 16 hours.

Cells are preincubated for 1 hour with either 20 μl serum-free medium (control), 20 μl 2,5 μM FFR-rFVIIa (control), 20 μl 2,5 μM anti TF Mab B or 20 μl 2,5 μM anti TF Mab A. 20 μl 0,5 μM FVIIa is added to half of the wells and medium to the other half. Following 4 hours of incubation the cells are subjected to the Luciferase gene assay. LucLite (Packard) reagent is added to the cells as described by the manufacturer. Luciferase expression levels are read on a TopCount Microplate Scintillation (Packard).

1. A method for preparing a human antibody comprising:
(a) immunizing a mouse capable of producing human antibodies with human tissue factor (“TF”),
(b) isolating antibody-producing cells from the immunized mouse and preparing immortal cells that secrete human antibodies therefrom,
(c) isolating culture medium from the immortal cells, wherein the culture medium comprises antibodies produced by the immortal cells,
(d) testing at least some of the antibodies in an indirect TF ELISA comprising TF in solution,
(e) testing at least some of the antibodies in a FVIIa competition assay,
(f) testing at least some of the antibodies in a FVIIa/TF amidolytic assay,
(g) testing at least some of the antibodies in a FXa generation assay,
(h) testing at least some of the antibodies in a TF-induced clot assay, and
(i) selecting at least one antibody from the antibodies that immunoreacts with
human TF in solution in the TF ELISA; competes with FVIIa in the FVIIa competition assay; inhibits TF-induced amidolytic activity, with an IC₅₀ value lower than the IC₅₀ value of FFR-rFVIIa+100 nM in the FVIIa/TF amidolytic assay (using 10 nm FVIIa in the assay); inhibits FXa generation in the FXa generation assay with an IC₅₀ value lower than the IC₅₀ value of FFR-rFVIIa+100 nM (using 0.1 nM FVIIa in the assay); and inhibits clot formation in the TF-induced clot assay with an IC₅₀ value lower than the IC₅₀ value of FFR-rFVIIa+1 nMo.

2. The method of claim 1, wherein the method comprises selecting at least one antibody that inhibits TF-induced FVIIa amidolytic activity with an IC₅₀ value of less than the IC₅₀ value of FFR-rFVIIa+40 nM.

3. The method of claim 2, wherein the method comprises selecting at least one antibody that inhibits TF-induced FVIIa amidolytic activity with an IC₅₀ value of less than the IC₅₀ value of FFR-rFVIIa+20 nM.

4. The method of claim 3, wherein the method comprises selecting at least one antibody that inhibits TF-induced FVIIa amidolytic activity with an IC₅₀ value of less than the IC₅₀value of FFR-rFVIIa+10 nM

5. The method of claim 4, wherein the method comprises selecting at least one antibody that inhibits TF-induced FVIIa amidolytic activity with an IC₅₀ value of less than the IC₅₀ value of FFR-rFVIIa

6. The method of claim 1, wherein the method comprises selecting at least one antibody that inhibits FXa generation with an IC₅₀ value of less than the IC₅₀ value of FFR-rFVIIa+10 nM.

7. The method of claim 6, wherein the method comprises selecting at least one antibody that inhibits FXa generation with an IC₅₀ value of less than the IC₅₀ value of FFR-rFVIIa+5 nM.

8. The method of claim 7, wherein the method comprises selecting at least one antibody that inhibits FXa generation with an IC₅₀ value of less than the IC₅₀ value of FFR-rFVIIa+0.1 nM.

9. The method of claim 8, wherein the method comprises selecting at least one antibody that inhibits FXa generation with an IC₅₀ value of less than the IC₅₀ value of FFR-rFVIIa.

10. The method of claim 1, wherein the method comprises selecting at least one antibody that inhibits clot formation in the TF-induced clot formation assay with an IC₅₀ value that is less than the IC₅₀ value of FFR-rFVIIa+500 pM.

11. The method of claim 10, wherein the method comprises selecting at least one antibody that inhibits clot formation in the TF-induced clot formation assay with an IC₅₀ value that is less than the IC₅₀ value of FFR-rFVIIa+50 pM.

12. The method of claim 11, wherein the method comprises selecting at least one antibody that inhibits clot formation in the TF-induced clot formation assay with an IC₅₀ value that is less than the IC₅₀ value of FFR-rFVIIa.

13. The method of claim 1, wherein the method further comprises testing at least some of the antibodies in a direct TF ELISA assay comprising immobilized TF and selecting at least one antibody which immunoreacts with immobilized human TF in the assay.

14. The method of claim 1, wherein the method further comprises testing at least some of the antibodies in a FXa generation assay on TF expressing cells and selecting at least one antibody which inhibits FXa generation on TF expressing cells with an IC₅₀ value that is less than the IC₅₀ value of FFR-rFVIIa+500 nM (using 1 nM FVIIa in the assay).

15. The method of claim 1, wherein the method further comprises testing at least some of the antibodies in a whole cell TF binding assay and selecting at least one antibody which competes with FVIIa binding to human TF expressed on the cell surface of whole cells.

16. The method of claim 1, wherein the method further comprises testing at least some of the antibodies in a biosensor assay and selecting at least one antibody with a K_{d} for binding to human TF that is less than 100 nM.

17. The method of claim 1, wherein the method further comprises testing at least some of the antibodies in a MAPK signalling assay and selecting at least one antibody which inhibits FVIIa-induced activation of the MAPK signalling in the assay.

18. The method of claim 1, wherein the method further comprises testing at least some of the antibodies in an epitope mapping assay and selecting at least one antibody which immunoreacts with preferred epitopes on TF.

19. The method of claim 18, wherein the preferred epitopes comprise residues Trp45, Lys46, and Tyr94.

20. The method of claim 1, wherein the method comprises selecting a cell that produces a selected antibody, culturing the cell, and obtaining isolated antibody from the culture.
